(19) United States
(12) Patent Application Publication
(10) Pub. No.: US 2022/0290136 A1
(43) Pub. Date: Sep. 15, 2022

(54) MATERIALS AND METHODS FOR TREATMENT OF AMYOTROPHIC LATERAL SCLEROSIS

(71) Applicant: CRISPR THERAPEUTICS AG, Zug (CH)

(72) Inventors: Hari Kumar Padmanabhan, Zug (CH); Adam James Donoghue, Zug (CH)

(21) Appl. No.: 17/489,218

(22) Filed: Sep. 29, 2021

Related U.S. Application Data
(60) Provisional application No. 63/085,636, filed on Sep. 30, 2020.

(51) Int. Cl. C12N 15/113

(52) U.S. Cl. CPC C12N 15/113; C12N 2310/20; C12N 2320/11

(57) ABSTRACT

The present application provides materials and methods for treating a patient with Amyotrophic Lateral Sclerosis (ALS). In addition, the present application provides materials and methods for (1) modifying the transcription start site of exon1a to render the transcription start site non-functioning, (2) deleting the transcription site of exon1a, (3) deleting exon1a, or (4) deleting of the expanded hexanucleotide repeat within or near the C9ORF72 gene, or any combinations of (1)-(4), above in a cell by genome editing.

### CROSS-REFERENCE TO RELATED APPLICATIONS

The present application claims the benefit of priority to U.S. Provisional Application No. 63/085,636, filed Sep. 30, 2020, the disclosure of which is incorporated herein by reference in their entirety.

### FIELD

The present application provides materials and methods for treating a patient with Amyotrophic Lateral Sclerosis (ALS). In addition, the present application provides materials and methods for editing to delete the expanded hexanucleotide repeat of the C9ORF72 gene in a cell by genome editing.

### INCORPORATION BY REFERENCE OF SEQUENCE LISTING

This application contains a Sequence Listing in computer readable form (filename: CT145_SeqListing.txt; 12,121 bytes—ASCII text file; created Sep. 28, 2021), which is incorporated herein by reference in its entirety and forms part of the disclosure.

### BACKGROUND

Amyotrophic lateral sclerosis (ALS) is a fatal neurodegenerative disease characterized clinically by progressive paralysis leading to death from respiratory failure, typically within two to three years of symptom onset (Rowland and Shneider, N. Engl. J. Med., 2001, 344, 1688-1700). ALS is the third most common neurodegenerative disease in the Western world (Hirtz et al., Neurology, 2007, 68, 326-337). Approximately 10% of cases are familial in nature, whereas the bulk of patients diagnosed with the disease are classified as sporadic, as they appear to occur randomly throughout the population (Chio et al., Neurology, 2008, 70, 533-537). There is growing recognition, based on clinical, genetic, and epidemiological data that ALS and Frontotemporal Lobular Dementia represent an overlapping continuum of disease, characterized pathologically by the presence of TDP-43 positive inclusions throughout the central nervous system (Lillo and Hodges, J. Clin. Neurosci, 2009, 16, 1131-1135; Neumann et al., Science, 2006, 314, 130-133).

To date, a number of genes have been discovered as causative for classical familial ALS, for example, SOD1, TARDBP, FUS, OPTN, and VCP (Johnson et al., Neuron, 2010, 68, 857-864; Kwiatkowski et al., Science, 2009, 323, 1205-1208; Maruyama et al., Nature, 2010, 465, 223-226; Rosen et al., Nature, 1993, 362, 59-62; Sreedharan et al., Science, 2008, 319, 1668-1672; Vance et al., Brain, 2009, 129, 868-876). Over the past 10 years, linkage analysis of kindreds involving multiple cases of ALS, FTD, and ALS-FTD identified an important locus for the disease on the short arm of chromosome 9, which is now known as C9orf72 (Boxer et al., J. Neurol. Neurosurg. Psychiatry, 2011, 82, 196-203; Morita et al., Neurology, 2006, 66, 839-844; Pearson et al. J. Neurol., 2011, 258, 647-655; Vance et al., Brain, 2006, 129, 868-876).

Currently, there are two FDA approved drugs on the market for the treatment of ALS, RILUTEK (riluzole) and RADACAVA (edaravone). However, the mechanism of action is poorly understood. RILUTEK and RADICAVA modestly slow the disease's progression in some people by reducing levels of glutamate in the brain and by reducing oxidative stress, respectively.

C9orf72 (chromosome 9 open reading frame 72) is a protein which, in humans, is encoded by the gene C9ORF72. The human C9ORF72 gene is located on the short (p) arm of chromosome 9 open reading frame 72, from base pair 27,546,542 to base pair 27,573,863. Its cytogenetic location is at 9p21.2. The protein is found in many regions of the brain, in the cytoplasm of neurons, as well as in presynaptic terminals. Disease causing mutations in the gene were first discovered by two independent research teams in 2011 (DeJesus-Hernandez et al. (2011) Neuron 72 (2): 245-56; Renton et al. (2011). Neuron 72 (2): 257-68). The mutation in C9ORF72 is significant because it is the first pathogenic mechanism identified to be a genetic link between FTLD and ALS. As of 2020, it is the most common mutation identified that is associated with familial FTLD and/or ALS.

The mutation of C9ORF72 is a hexanucleotide repeat expansion (HRE) of the six letter string of nucleotides GGGGCC. In healthy individuals, there are few repeats of this hexanucleotide, typically 30, but in people with the diseased phenotype, the repeat can occur in the order of hundreds (Fong et al. (2012) Alzheimers Res Ther 4 (4): 27). The hexanucleotide expansion event in the C9ORF72 gene is present in approximately 40% of familial ALS and 8-10% of sporadic ALS patients. The hexanucleotide expansion occurs in an alternatively spliced Intron 1 of the C9ORF72 gene, and as such does not alter the coding sequence or resulting protein. Three alternatively spliced variants of C9ORF72 (V1, V2 and V3) are normally produced. The expanded nucleotide repeat was shown to reduce the transcription of V1, however the total amount of protein produced was unaffected (DeJesus-Hernandez et al. (2011), Neuron 72 (2): 245-56). Overall, reduced protein levels of C9ORF72 have been observed in brain autopsies from ALS patients (Waite (2014) Neurobiol Aging, 35 1779 e1775-1779 e1713) suggesting haploinsufficiency as a cause of ALS/FTD.

In addition to haploinsufficiency, there are other theories about the way in which the C9ORF72 hexanucleotide expansion causes FTD and/or ALS. Another theory is that accumulation of GC rich RNA in the nucleus and cytoplasm becomes toxic, and RNA binding protein sequestration occurs. A common feature of non-coding repeat expansion disorders, which has gained increased attention in recent years, is the accumulation of RNA fragments composed of the repeated nucleotides as RNA foci in the nucleus and/or cytoplasm of affected cells (Todd and Paulson, 2010, Ann. Neurol. 67, 291-300). In several disorders, the RNA foci have been shown to sequester RNA-binding proteins, leading to dysregulation of alternative mRNA splicing. A hallmark of C9ORF72ALS is cytoplasmic inclusions of an RNA binding protein TDP-43 throughout the central nervous system (Lillo and Hodges, J. Clin. Neurosci, 2009, 16, 1131-1135; Neumann et al., Science, 2006, 314, 130-133).

An additional theory is that RNA transcribed from the C9ORF72 gene containing expanded hexanucleotide repeats is translated through a non-ATG initiated mechanism. This drives the formation and accumulation of dipeptide repeat proteins corresponding to multiple ribosomal reading frames on the mutation. The repeat is translated into dipeptide repeat (DPR) proteins that cause repeat-induced toxicity. DPRs inhibit the proteasome and sequester other proteins. GGGGCC repeat expansion in C9ORF72 may compromise nucleocytoplasmic transport through several possible mechanisms (Edbauer, Current Opinion in Neurobiology 2016, 36:99-106).

Traditionally, familial and sporadic cases of ALS have been clinically indistinguishable, which has made diagnosis difficult. The identification of this gene will therefore help in the future diagnosis of familial ALS. Slow diagnosis is also common for FTD, which can often take up to a year with many patients initially misdiagnosed with another condition. Testing for a specific gene that is known to cause the diseases would help with faster diagnoses. Most importantly, this hexanucleotide repeat expansion is an extremely promising future target for developing therapies to treat both familial FTD and familial ALS.

Genome engineering refers to the strategies and techniques for the targeted, specific modification of the genetic information (genome) of living organisms. Genome engineering is a very active field of research because of the wide range of possible applications, particularly in the areas of human health; the correction of a gene carrying a harmful mutation, for example, or to explore the function of a gene. Early technologies developed to insert a transgene into a living cell were often limited by the random nature of the insertion of the new sequence into the genome. Random insertions into the genome may result in disrupting normal regulation of neighboring genes leading to severe unwanted effects. Furthermore, random integration technologies offer little reproducibility, as there is no guarantee that the sequence would be inserted at the same place in two different cells. Recent genome engineering strategies, such as ZFNs, TALENs, HEs and MegaTALs, enable a specific area of the DNA to be modified, thereby increasing the precision of the correction or insertion compared to early technologies. These newer platforms offer a much larger degree of reproducibility.

Despite efforts from researchers and medical professionals worldwide who have been trying to address ALS, and despite the promise of genome engineering approaches, there still remains a critical need for developing safe and effective treatments for ALS.

### SUMMARY

In one aspect, described herein is a method for editing the C9ORF72 gene in a human cell by genome editing comprising introducing into the cell one or more deoxyribonucleic acid (DNA) endonucleases to effect one or more double-strand breaks (DSBs) within or near the first exon of the C9ORF72 gene that results in modification of exon1a transcription start site within the C9ORF72 gene. In some embodiments, the modification renders the transcription start site non-functional. a single DSB is targeting the transcription start site of exon1a. In some embodiments, the C9ORF72 gene is located on Chromosome 9: 27,546,542-27,573,863 (Genome Reference Consortium—GRCh38/hg38).

In another aspect, described herein is a method for editing the C9ORF72 gene in a human cell by genome editing comprising introducing into the cell one or more deoxyribonucleic acid (DNA) endonucleases to effect one or more double-strand breaks (DSBs) within or near the first exon of the C9ORF72 gene that results in deletion of exon1a transcription start site within the C9ORF72 gene. In some embodiments, the method results in deletion of exon1a of the C9ORF72 gene. In some embodiments, the method results in deletion of exon1a and expanded hexanucleotide repeat associated with ALS/FTD of the C9ORF72 gene.

In some embodiments, the one or more DSBs are upstream of the transcription start site of exon1a. In some embodiments, the one or more DSBs are within an upstream sequence region of the C9ORF72 gene. In some embodiments, the one or more DSBs are within 500 nucleotides of the transcription start site for exon1a. In some embodiments, the one or more DSBs are within at least 200 nucleotides of the transcription start site for exon1a. In some embodiments, the one or more DSBs are within at least 10, 15, 20, 25, 30, 35, 40, 45, 50, 55, 60, 65, 70, 75, 80, 85, 90, 95, 100, 110, 120, 125, 130, 135, 140, 145, 150, 155, 160, 165, 170, 175, 180, 185, 190, 195, 200, 250, 300, 350, 400, 450 or 500 nucleotides of the transcriptional start site for exon1a.

In some embodiments, a first DSB is upstream of the transcription start site of exon1a and a second DSB is downstream of the transcription start site of exon1a.

In some embodiments, a first DSB is upstream of the transcription start site of exon1a and a second DSB is in exon1a downstream of the transcription start site of exon1a. In some embodiments, a first DSB is upstream of the transcription start site of exon1a and a second DSB is in intron 1 and upstream of the hexanucleotide repeat. In some embodiments, a first DSB is upstream of the transcription start site of exon1a and a second DSB is in intron 1 and downstream of the hexanucleotide repeat.

In another aspect, described herein is method for editing the C9ORF72 gene in a human cell by genome editing comprising introducing into the cell one or more deoxyribonucleic acid (DNA) endonucleases to effect one or more double-strand breaks (DSBs) within or near the hexanucleotide repeat of the C9ORF72 gene that results in deletion of hexanucleotide repeat within the C9ORF72 gene. In some embodiments, the expanded hexanucleotide repeat is within the first intron of the C9ORF72 gene. In some embodiments, a first DSB is upstream of the hexanucleotide repeat of the first intron of the C9ORF72 gene. and the second DSB is downstream of the hexanucleotide repeat of the first intron of the C9ORF72 gene.

In some embodiments, the one or more DNA endonucleases is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 (also known as Cas12a) endonuclease; or a homolog thereof, recombination of the naturally occurring molecule, codon-optimized, or modified version thereof, and combinations thereof.

In various embodiments, the methods described herein comprise introducing into the cell one or more polynucleotides encoding the one or more DNA endonucleases. In some embodiments, the one or more polynucleotides or one or more RNAs is one or more modified polynucleotides or one or more modified RNAs.

The methods described herein optionally further comprise introducing into the cell one or more guide ribonucleic acids (gRNAs). In some embodiments, the one or more gRNAs are single-molecule guide RNA (sgRNAs). In some embodiments, the one or more DNA endonucleases is pre-complexed with one or more gRNAs or one or more sgRNAs.

In some embodiments, the methods described herein comprise introducing into the cell a guide ribonucleic acid (gRNA), and wherein the DNA endonucleases is a Cas9 or Cpf1 endonuclease that effect a single double-strand breaks (DSBs) within the transcription start site of exon1a of the C9ORF72 gene that renders the transcription start site to be non-functional.

In some embodiments, the methods described herein comprise introducing into the cell two guide ribonucleic acid (gRNAs), and wherein the one or more site-directed DNA endonucleases is two or more Cas9 or Cpf1 endonucleases that effect a pair of double-strand breaks (DSBs), the first DSB is at a 5′ locus of the exon1a transcription start site of the C9ORF72 gene and the second DSB is at a 3′ locus of the exon1a transcription start site that causes a permanent deletion of the exon1a transcription start site of the C9ORF72 gene.

In some embodiments, the methods described herein comprise introducing into the cell two guide ribonucleic acid (gRNAs), and wherein the one or more site-directed DNA endonucleases is two or more Cas9 or Cpf1 endonucleases that effect a pair of double-strand breaks (DSBs), the first DSB is at a 5′ locus of the exon1a transcription start site of the C9ORF72 gene and a second DSB that is 3′ of intron 1 but upstream of the hexanucleotide repeat of the C9ORF72 gene that causes a permanent deletion of the exon1a of the C9ORF72 gene.

In some embodiments, the methods described herein comprise introducing into the cell two guide ribonucleic acid (gRNAs), and wherein the one or more site-directed DNA endonucleases is two or more Cas9 or Cpf1 endonucleases that effect a pair of double-strand breaks (DSBs), the first DSB is at a 5′ locus of the exon1a transcription start site of the C9ORF72 gene and a second DSB that is 3′ of intron 1 but downstream of the hexanucleotide repeat of the C9ORF72 gene that causes a permanent deletion of the hexanucleotide repeat of the C9ORF72 gene.

In some embodiments, the methods described herein comprise introducing into the cell two guide ribonucleic acid (gRNAs), and wherein the one or more site-directed DNA endonucleases is two or more Cas9 or Cpf1 endonucleases that effect a pair of double-strand breaks (DSBs), the first DSB is at a 5′ locus upstream of the hexanucleotide repeat in intron 1 of the C9ORF72 gene and a second DSB that is 3′ of intron 1 but downstream of the hexanucleotide repeat of the C9ORF72 gene that causes a permanent deletion of the hexanucleotide repeat of the C9ORF72 gene.

In some embodiments, the one or more gRNAs comprises a nucleotide sequence set forth in SEQ ID NOs: 1-9. In some embodiments, the two gRNAs are set forth in (a) SEQ ID NOs: 1 and 2 (T11 and T7); (b) SEQ ID NOs: 3 and 4 (T3 and T62); (c) SEQ ID NOs: 5 and 2 (T30 and T7); (d) SEQ ID NOs: 5 and 4 (T30 and T62); (e) SEQ ID NOs: 1 and 6 (T11 and T69); (f) SEQ ID NOs: 3 and 6 (T3 and T69); (g) SEQ ID NOs: 5 and 6 (T30 and T69); (h) SEQ ID NOs: 3 and 7 (T3 and T118); (i) SEQ ID NOs: 5 and 7 (T30 and T118); (j) SEQ ID NOs: 1 and 8 (T11 and T118); (k) SEQ ID NOs: 8 and 7 (T17 and T118); or (l) SEQ ID NOs: 9 and 6 (T128 and T69). In some embodiments, the two gRNAs are set forth in (a) SEQ ID NOs: 1 and 2 (T11 and T7); (b) SEQ ID NOs: 3 and 4 (T3 and T62); (c) SEQ ID NOs: 5 and 2 (T30 and T7); or (d) SEQ ID NOs: 5 and 4 (T30 and T62). In some embodiments, the two gRNAs are set forth in (a) SEQ ID NOs: 1 and 6 (T11 and T69); (b) SEQ ID NOs: 3 and 6 (T3 and T69); (c) SEQ ID NOs: 5 and 6 (T30 and T69); (d) SEQ ID NOs: 3 and 7 (T3 and T118); (e) SEQ ID NOs: 5 and 7 (T30 and T118); (f) SEQ ID NOs: 1 and 7 (T11 and T118); or (g) SEQ ID NOs: 8 and 7 (T17 and T118). In some embodiments, the two gRNAs are SEQ ID NO: 9 and SEQ ID NO: 6 (T128 and T69).

In some embodiments, the Cas9 or Cpf1 mRNA and gRNA are either each formulated separately into lipid nanoparticles or all co-formulated into a lipid nanoparticle. In other embodiments, the Cas9 or Cpf1 mRNA is formulated into a lipid nanoparticle, and the gRNA is delivered by a viral vector. In some embodiments, the viral vector is an adeno-associated virus (AAV) vector (e.g., AAV9).

In some embodiments, the Cas9 or Cpf1 mRNA are delivered by a viral vector and the gRNA is delivered by the same or an additional viral vector. In some embodiments, the viral vector is an adeno-associated virus (AAV) vector (e.g., AAV9).

In some embodiments, the Cas9 or Cpf1 mRNA and gRNA are either each formulated into separate exosomes or all co-formulated into an exosome.

In any of the embodiments, the methods described herein result in a reduction in hexanucleotide repeat containing transcripts of C9ORF72 is observed compared to wild-type C9ORF72 gene transcripts. In some embodiments, the methods described herein result in an at least 10% (e.g., at least 10%, 15%, 20%, 25%, 30%, 40% or more) reduction in expanded hexanucleotide repeat containing transcripts of C9ORF72 compared to wild-type C9ORF72 gene transcripts.

In another aspect, described herein is a method for editing a C9ORF72 gene in a human cell by gene editing comprising delivering to the cell one or more CRISPR systems comprising one or more guide ribonucleic acids (gRNAs) and one or more site-directed deoxyribonucleic acid (DNA) endonucleases, and wherein the one or more DNA enconucleases are Cas9 endonucleases that effect double-stranded breaks (DSBs) within a region of the C9ORF72 gene comprising nucleotides 1801-2900 of SEQ ID NO: 42 that causes a permanent deletion of the hexanucleotide repeat of the C9ORF72 gene.

In some embodiments, the region of the C9ORF72 gene comprises nucleotides 1801-1970 of SEQ ID NO: 42. In some embodiments, the region of the C9ORF72 gene comprises nucleotides 2051-2156 of SEQ ID NO: 42. In some embodiments, the region of the C9ORF72 gene comprises nucleotides 2189-2326 of SEQ ID NO: 42. In some embodiments, the region of the C9ORF72 gene comprises nucleotides 2384-2900 of SEQ ID NO: 42.

In some embodiments, a first DSB is within nucleotides 1801-1970 of SEQ ID NO: 42 and a second DSB is within nucleotides 2051-2156 of SEQ ID NO: 42. In some embodiments, a first DSB is within nucleotides 1801-1970 of SEQ ID NO: 42 and a second DSB is within nucleotides 2189-2326 of SEQ ID NO: 42. In some embodiments, a first DSB is within nucleotides 1801-1970 of SEQ ID NO: 39 and a second DSB is within nucleotides 2384-2900 of SEQ ID NO: 42.

In another aspect, described herein are one or more guide ribonucleic acids (gRNAs) comprising a spacer sequence selected from the nucleotide sequence set forth in SEQ ID NOs.: 1-41. In some embodiments, the one or more guide ribonucleic acids (gRNAs) comprising a spacer sequence set forth in SEQ ID NOs: 1, 2, 3, 4, 5, 6, 7, 8, 9, and 15. In some embodiments, the one or more guide ribonucleic acids (gRNAs) comprising a spacer sequence set forth in SEQ ID NOs: 1, 2, 3, 4, 5, 6, 7, 8, 9, 15, 17, 18, 20, 21, 26, 31, 33, 34, and 40.

In some embodiments, the one or more gRNAs are (a) SEQ ID NO: 1 and SEQ ID NO: 2 (T1 and T7); (b) SEQ ID NO: 1 and SEQ ID NO: 7 (T1 and T118); (c) SEQ ID NO: 1 and SEQ ID NO: 6 (T1 and T69); (d) SEQ ID NO: 8 and SEQ ID NO: 7 (T17 and T118; (e) SEQ ID NO: 1 and SEQ ID NO: 15 (T1 and T5); (f) SEQ ID NO: 3 and SEQ ID NO: 7 (T3 and T118); (g) SEQ ID NO: 3 and SEQ ID NO: 15 (T3 and T5); (h) SEQ ID NO: 3 and SEQ ID NO: 6 (T3 and T69); (i) SEQ ID NO: 5 and SEQ ID NO: 2 (T30 and T7); (j) SEQ ID NO: 5 and SEQ ID NO: 7 (T30 and T118); (k) SEQ ID NO: 5 and SEQ ID NO: 15 (T30 and T5); (l) SEQ ID NO: 5 and SEQ ID NO: 6 (T30 and T69); (m) SEQ ID NO: 9 and SEQ ID NO: 6 (T128 and T69); or (n) SEQ ID NO: 5 and SEQ ID NO: 4 (T30 and T62).

In some embodiments, the one ore more gRNAs are (a) SEQ ID NO: 20 and SEQ ID NO: 21 (S2 and S24); (b) SEQ ID NO: 20 and SEQ ID NO: 22 (S2 and S31); (c) SEQ ID NO: 26 and SEQ ID NO: 18 (S17 and S26); (d) SEQ ID NO: 26 and SEQ ID NO: 29 (S28 and S29); (e) SEQ ID NO: 41 and SEQ ID NO: 24 (51 and S22); (f) SEQ ID NO: 20 and SEQ ID NO: 34 (S2 and S9); (g) SEQ ID NO: 17 and SEQ ID NO: 33 (S3 and S6); or (h) SEQ ID NO: 20 and SEQ ID NO: 33 (S2 and S6).

In some embodiments, the one or more gRNAs are (a) SEQ ID NO: 6 and SEQ ID NO: 21 (S2 and S24), (b) SEQ ID NO: 6 and SEQ ID NO: 22 (S2 and S31), (c) SEQ ID NO: 6 and SEQ ID NO: 33 (S2 and S6), (d) SEQ ID NO: 6 and SEQ ID NO: 34 (S2 and S9), (e) SEQ ID NO: 17 and SEQ ID NO: 33 (S3 and S6), (f) SEQ ID NO: 26 and SEQ ID NO: 18 (S17 and S26), or (g) SEQ ID NO: 31 and SEQ ID NO: 40 (S28 and S29).

In some embodiments, the one or more gRNAs are one or more single-molecule guide RNAs (sgRNAs). In some embodiments, the one or more gRNAs or one or more sgRNAs is one or more modified gRNAs or one or more modified sgRNAs.

The disclosure also provides a recombinant expression vector comprising a nucleotide sequence that encodes the one or more gRNAs described herein. In some embodiments, the vector is a viral vector. In some embodiments, the viral vector is an adeno-associated virus (AAV) vector. In some embodiments, the vector comprises a nucleotide sequence encoding a Cas9 DNA endonuclease. In some embodiments, the Cas9 endonuclease is a SpCas9 endonuclease. In some embodiments, the Cas9 endonuclease is a SluCas9 endonuclease. In some embodiments, the vector is formulated in a lipid nanoparticle.

The disclosure also provides a pharmaceutical composition comprising the one or more gRNAs described herein or vector described herein and a pharmaceutically acceptable carrier.

In another aspect, the disclosure provides a system for introducing a deletion of the hexanucleotide repeat of the C9ORF72 gene in a cell, the system comprising: (i) one or more site-directed DNA enconucleases; and (ii) one or more ribonucleic acids (gRNAs) comprising a spacer sequence corresponding to a target sequence within nucleotides 1801-2900 of SEQ ID NO: 42, wherein when the one or more gRNAs is introduced to the cell with the DNA endonucleases, the one or more gRNAs combine with the DNA endonuclease to induce double-stranded breaks (DSBs) within a region of the C9ORF72 gene comprising nucleotides 1801-2900 of SEQ ID NO: 42. In some embodiments, the one or more DNA endonucleases is a Cas9 endonuclease. In some embodiments, the Cas9 endonuclease is a SpCas9 polypeptide, an mRNA encoding the SpCas9 polypeptide, or a recombinant expression vector comprising a nucleotide sequence encoding the SpCas9 polypeptide. In some embodiments, the Cas9 endonuclease is a SluCas9 polypeptide, an mRNA encoding the SluCas9 polypeptide, or a recombinant expression vector comprising a nucleotide sequence encoding the SluCas9 polypeptide.

In some embodiments, the region of the C9ORF72 gene comprises nucleotides 1801-1970 of SEQ ID NO: 42. In some embodiments, the region of the C9ORF72 gene comprises nucleotides 2051-2156 of SEQ ID NO: 42. In some embodiments, the region of the C9ORF72 gene comprises nucleotides 2189-2326 of SEQ ID NO: 42. In some embodiments, the region of the C9ORF72 gene comprises nucleotides 2384-2900 of SEQ ID NO: 42.

In some embodiments, a first DSB is within nucleotides 1801-1970 of SEQ ID NO: 42 and a second DSB is within nucleotides 2051-2156 of SEQ ID NO: 42. In some embodiments, a first DSB is within nucleotides 1801-1970 of SEQ ID NO: 42 and a second DSB is within nucleotides 2189-2326 of SEQ ID NO: 42. In some embodiments, a first DSB is within nucleotides 1801-1970 of SEQ ID NO: 42 and a second DSB is within nucleotides 2384-2900 of SEQ ID NO: 42.

In some embodiments, the one or more gRNAs are: (a) SEQ ID NO: 1 and SEQ ID NO: 2 (T1 and T7); (b) SEQ ID NO: 1 and SEQ ID NO: 7 (T1 and T118); (c) SEQ ID NO: 1 and SEQ ID NO: 6 (T1 and T69); (d) SEQ ID NO: 8 and SEQ ID NO: 7 (T17 and T118); (e) SEQ ID NO: 1 and SEQ ID NO: 15 (T1 and T5); (f) SEQ ID NO: 3 and SEQ ID NO: 7 (T3 and T118); (g) SEQ ID NO: 3 and SEQ ID NO: 15 (T3 and T5); (h) SEQ ID NO: 3 and SEQ ID NO: 6 (T3 and T69); (i) SEQ ID NO: 5 and SEQ ID NO: 2 (T30 and T7); (j) SEQ ID NO: 5 and SEQ ID NO: 7 (T30 and T118); (k) SEQ ID NO: 5 and SEQ ID NO: 15 (T30 and T5); (l) SEQ ID NO: 5 and SEQ ID NO: 6 (T30 and T69); (m) SEQ ID NO: 9 and SEQ ID NO: 6 (T128 and T69); or (n) SEQ ID NO: 5 and SEQ ID NO: 4 (T30 and T62).

In some embodiments, the one or more gRNAs are: (a) SEQ ID NO: 20 and SEQ ID NO: 21 (S2 and S24); (b) SEQ ID NO: 20 and SEQ ID NO: 22 (S2 and S31); (c) SEQ ID NO: 26 and SEQ ID NO: 18 (S17 and S26); (d) SEQ ID NO: 26 and SEQ ID NO: 29 (S28 and S29); (e) SEQ ID NO: 41 and SEQ ID NO: 24 (51 and S22); (f) SEQ ID NO: 20 and SEQ ID NO: 34 (S2 and S9); (g) SEQ ID NO: 17 and SEQ ID NO: 33 (S3 and S6); or (h) SEQ ID NO: 20 and SEQ ID NO: 33 (S2 and S6).

In some embodiments, the one or more gRNAs are: (a) SEQ ID NO: 20 and SEQ ID NO: 21 (S2 and S24), (b) SEQ ID NO: 20 and SEQ ID NO: 22 (S2 and S31), (c) SEQ ID NO: 20 and SEQ ID NO: 33 (S2 and S6), (d) SEQ ID NO: 20 and SEQ ID NO: 34 (S2 and S9), (e) SEQ ID NO: 17 and SEQ ID NO: 33 (S3 and S6), (f) SEQ ID NO: 26 and SEQ ID NO: 18 (S17 and S26), or (g) SEQ ID NO: 31 and SEQ ID NO: 40 (S28 and S29).

In some embodiments, the system comprises a recombinant expression vector comprises (i) a nucleotide sequence encoding a site-directed DNA endonuclease and (ii) a nucleotide sequence encoding the one or more gRNAs. In some embodiments, the system comprises a first recombinant expression vector comprising a nucleotide sequence encoding the site-directed DNA endonuclease and a second recombinant expression vector comprising a nucleotide sequence encoding the one or more gRNA. In some embodiments, the vector is a viral vector. In some embodiments, the viral vector is an adeno-associated viral (AAV) vector. In some embodiments, the AAV vector is AAV9.

In some embodiments, the site-directed DNA endonuclease and gRNA are either each formulated separately into lipid nanoparticles or all co-formulated into a lipid nanoparticle. In other embodiments, the site-directed DNA endonuclease is formulated into a lipid nanoparticle, and the gRNA is delivered by a viral vector. In some embodiments, the viral vector is an adeno-associated virus (AAV) vector (e.g., AAV9).

### BRIEF DESCRIPTION OF THE DRAWINGS

FIGS. 1A-1C provide schematics of the C9ORF72 locus and transcription. FIG. 1A shows the C9ORF72 gene Locus. The Hexanucleotide Repeat Expansion (HRE) is situated between to variants of Exon1. FIG. 1B shows that the HRE uses the same transcription start site as Exon1a. FIG. 1C shows that the presence of HRE leads to heterochromatin restructuring that blocks transcription of the major isoform leading to haploinsufficiency of C9ORF72.

FIG. 2 is a schematic showing C9ORF72 genome editing strategies.

FIG. 3 provides graphs showing that guide RNA pairs T11/T7 and T17/T62 that delete regions upstream of G4C2 repeats (that included Exon1a) caused a dramatic reduction in expression of Exon1a and HRE-RNA.

FIG. 4 provides graphs showing that guide RNA pairs T128/T69 and T30/T69 that delete the G4C2 repeats caused a significant reduction in HRE-RNA levels.

FIG. 5 provides graphs showing that guide RNA pairs T132/T44 and T132/T9 that delete a potential regulatory region on the 3′ flank of the G4C2 repeats did not cause a reduction in HRE-RNA levels.

FIG. 6 is a table providing the guide RNA pairs assayed in Example 1.

FIGS. 7A and 7B are graphs showing that the level of C9ORF72 repeat containing transcripts in the tested clones was close to signal seen with Nanostring negative controls, demonstrating that deleting Exon1a from a C9ORF72 allele caused a complete loss of repeat expression from that allele and that these clones are homozygous for Exon1a deletion.

FIG. 8 provides a graph showing that guide pairs T11/T7 delete regions upstream of G4C2 repeats (that included Exon1a) caused a dramatic reduction in expression of Exon1a and HRE-RNA.

FIG. 9 provides a graph showing that Exon1A deletion correlates with a reduction in repeat containing transcripts.

FIG. 10 is a schematic showing the target regions for the SpCas9 guide pairs described in Example 1.

FIG. 11 is a schematic showing the target regions for the SluCas9 guide pairs described in Example 3.

### DETAILED DESCRIPTION

The human C9ORF72 gene is located on the short (p) arm of chromosome 9 open reading frame 72, from base pair 27,546,542 to base pair 27,573,863 (Genome Reference Consortium—GRCh38/hg38. Its cytogenetic location is at 9p21.2. The mutation of C9ORF72 is a hexanucleotide repeat expansion of the six letter string of nucleotides GGGGCC. In healthy individuals, there are few repeats of this hexanucleotide, typically 30 or less, but in people with the diseased phenotype, the repeat can occur in the order of hundreds. The hexanucleotide expansion event in the C9ORF72 gene is present in approximately 40% of familial ALS and 8-10% of sporadic ALS.

The hexanucleotide expansion occurs in an alternatively spliced Intron 1 of the C9ORF72 gene, and as such does not alter the coding sequence or resulting protein. Three alternatively spliced variants of C9ORF72 (V1, V2 and V3) are normally produced. The expanded nucleotide repeat has been shown to reduce the transcription of V1.

The term “hexanucleotide repeat expansion” or “HRE” means a series of six nucleotide bases (for example, GGGGCC, GGGGGG, GGGGCG, or GGGGGC) repeated at least twice. In certain embodiments, the hexanucleotide repeat expansion is located in intron 1 of a C9ORF72 nucleic acid. In certain embodiments, a pathogenic hexanucleotide repeat expansion (also referred to herein as an “expanded hexanucleotide repeat”) includes at least 23 repeats of GGGGCC, GGGGGG, GGGGCG, or GGGGGC in a C9ORF72 nucleic acid and is associated with disease (e.g., ALS). In other embodiments, a pathogenic hexanucleotide repeat expansion includes at least 23, 24, 25, 26, 27, 28, 29, 30, 31, 32, 33, 34, 35, 40, 45, 50, 60, 70, 80, 90, 100, 150, 200, 250, 300, 400, 500, 600, 700, 800, 900, 1000 or more repeats. In certain embodiments, the repeats are consecutive. In certain embodiments, the repeats are interrupted by 1 or more nucleobases. In certain embodiments, a wild-type hexanucleotide repeat expansion includes 22 or fewer repeats of GGGGCC, GGGGGG, GGGGCG, or GGGGGC in a C9ORF72 nucleic acid. In other embodiments, a wild-type hexanucleotide repeat expansion includes 22, 21, 20, 19, 18, 17, 16, 15, 14, 13, 12, 11, 10, 9, 8, 7, 6, 5, 4, 3, 2, or 1 repeat.

In one aspect, described herein is a method for editing the C9ORF72 gene in a human cell by genome editing comprising introducing into the cell one or more site-directed deoxyribonucleic acid (DNA) endonucleases to effect one or more double-strand breaks (DSBs) within or near the first exon of the C9ORF72 gene that results in modification of exon1a transcription start site within the C9ORF72 gene. In some embodiments, the modification renders the transcription start site non-functional. In some embodiments, the modification is a single DSB is targeting the transcription start site of exon1a.

In another aspect, described herein is a method for editing the C9ORF72 gene in a human cell by genome editing comprising introducing into the cell one or more site-directed deoxyribonucleic acid (DNA) endonucleases to effect one or more double-strand breaks (DSBs) within or near the first exon of the C9ORF72 gene that results in deletion of exon1a transcription start site within the C9ORF72 gene. In some embodiments, the method results in deletion of exon1a of the C9ORF72 gene. In some embodiments, the method results in deletion of exon1a and expanded hexanucleotide repeat associated with ALS/FTD of the C9ORF72 gene.

In some embodiments, the methods described herein comprise introducing into the cell a guide ribonucleic acid (gRNA), and wherein the site-directed DNA endonucleases is a Cas9 or Cpf1 endonuclease that effect a single double-strand breaks (DSBs) within the transcription start site of exon1a of the C9ORF72 gene that renders the transcription start site to be non-functional.

In some embodiments, the methods described herein comprise introducing into the cell two guide ribonucleic acid (gRNAs), and wherein the one or more site-directed DNA endonucleases is two or more Cas9 or Cpf1 endonucleases that effect a pair of double-strand breaks (DSBs), the first DSB is at a 5′ locus of the exon1a transcription start site of the C9ORF72 gene and the second DSB is at a 3′ locus of the exon1a transcription start site that causes a permanent deletion of the exon1a transcription start site of the C9ORF72 gene.

In some embodiments, the methods described herein comprise introducing into the cell two guide ribonucleic acid (gRNAs), and wherein the one or more site-directed DNA endonucleases is two or more Cas9 or Cpf1 endonucleases that effect a pair of double-strand breaks (DSBs), the first DSB is at a 5′ locus of the exon1a transcription start site of the C9ORF72 gene and a second DSB that is 3′ of intron 1 but upstream of the hexanucleotide repeat of the C9ORF72 gene that causes a permanent deletion of the exon1a of the C9ORF72 gene.

In some embodiments, the methods described herein comprise introducing into the cell two guide ribonucleic acid (gRNAs), and wherein the one or more site-directed DNA endonucleases is two or more Cas9 or Cpf1 endonucleases that effect a pair of double-strand breaks (DSBs), the first DSB is at a 5′ locus of the exon1a transcription start site of the C9ORF72 gene and a second DSB that is 3′ of intron 1 but downstream of the hexanucleotide repeat of the C9ORF72 gene that causes a permanent deletion of the hexanucleotide repeat of the C9ORF72 gene.

In some embodiments, the methods described herein comprise introducing into the cell two guide ribonucleic acid (gRNAs), and wherein the one or more DNA endonucleases is two or more Cas9 or Cpf1 endonucleases that effect a pair of double-strand breaks (DSBs), the first DSB is at a 5′ locus upstream of the hexanucleotide repeat in intron 1 of the C9ORF72 gene and a second DSB that is 3′ of intron 1 but downstream of the hexanucleotide repeat of the C9ORF72 gene that causes a permanent deletion of the hexanucleotide repeat of the C9ORF72 gene.

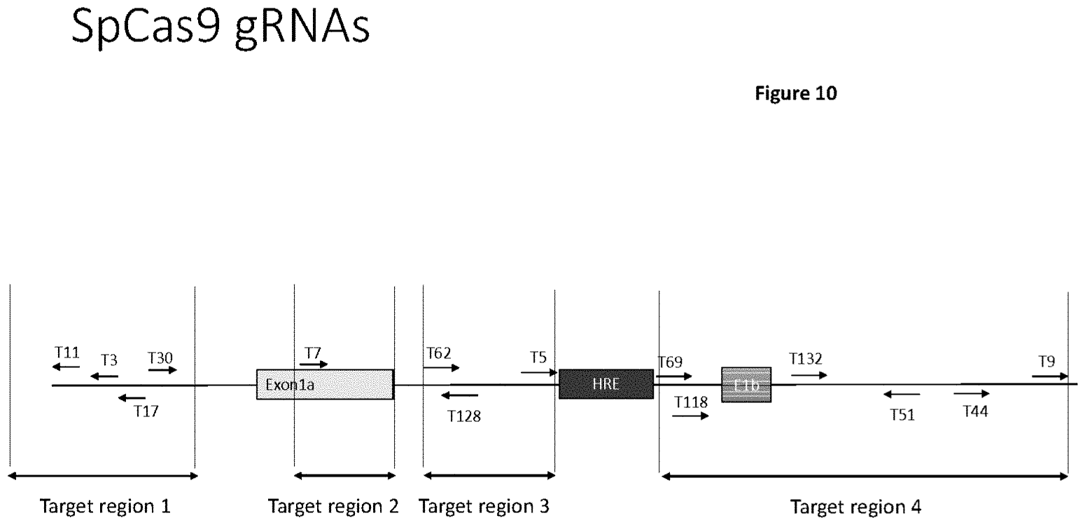

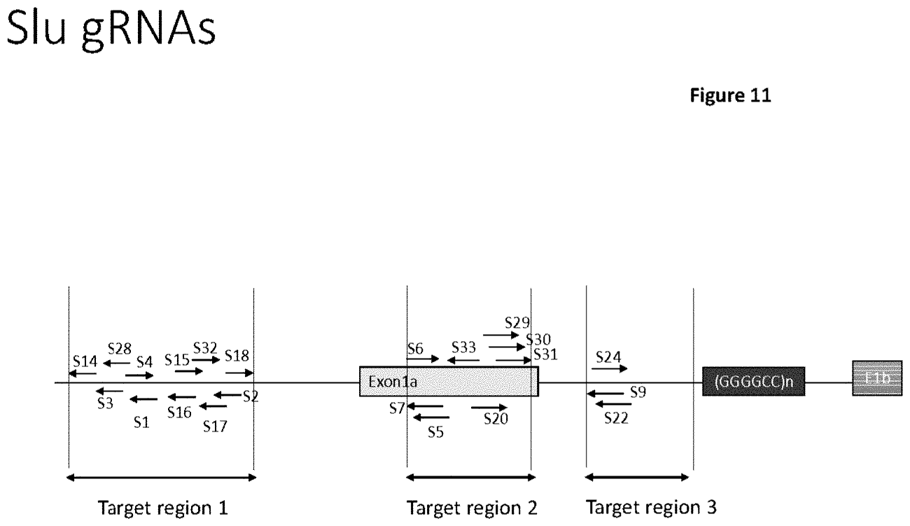

In some embodiments, the methods described herein comprise introducing into the cell two guide ribonucleic acids (gRNAs), and wherein the one or more DNA enconucleases is two or more Cas9 endonucleases that effect a pair of DSBs within a region the C9ORF72 gene comprising the nucleotide sequence set forth in SEQ ID NO: 42 that causes a permanent deletion of the hexanucleotide repeat of the C9ORF72 gene. In some embodiments, the region of the C9ORF72 gene comprises nucleotides 1801-2900 of SEQ ID NO: 42. In some embodiments, the region of the C9ORF72 gene comprises nucleotides 1801-1970 of SEQ ID NO: 42 (Target region 1 as shown in FIGS. 10 and 11). In some embodiments, the region of the C9ORF72 gene comprises nucleotides 2051-2156 of SEQ ID NO: 42 (Target region 2 as shown in FIGS. 10 and 11). In some embodiments, the region of the C9ORF72 gene comprises nucleotides 2189-2326 of SEQ ID NO: 42 (Target region 3 as shown in FIGS. 10 and 11). In some embodiments, the region of the C9ORF72 gene comprises nucleotides 2384-2900 of SEQ ID NO: 42 (Target region 4 as shown in FIG. 11).

In some embodiments, the region of the C9ORF72 gene comprises nucleotides 1801-1970 of SEQ ID NO: 42 and nucleotides 2051-2156 of SEQ ID NO: 42.

In some embodiments, the region of the C9ORF72 gene comprises nucleotides 1801-1970 of SEQ ID NO: 42 and nucleotides 2189-2326 of SEQ ID NO: 42.

In some embodiments, the region of the C9ORF72 gene comprises nucleotides 1801-1970 of SEQ ID NO: 42 and nucleotides 2384-2900 of SEQ ID NO: 42.

In another aspect, disclosed herein is a system for introducing a deletion of the hexanucleotide repeat of the C9ORF72 gene in a cell, the system comprising: (i) one or more site-directed DNA endonucleases; and (ii) one or more ribonucleic acids (gRNAs) comprising a spacer sequence corresponding to a target sequence within nucleotides 1801-2900 of SEQ ID NO: 42, wherein when the one or more gRNAs is introduced to the cell with the DNA endonucleases, the one or more gRNAs combine with the DNA endonuclease to induce double-stranded breaks (DSBs) within a region of the C9ORF72 gene comprising nucleotides 1801-2900 of SEQ ID NO: 42.

Genome Editing

Genome editing generally refers to the process of modifying the nucleotide sequence of a genome, preferably in a precise or pre-determined manner. Examples of methods of genome editing described herein include methods of using site-directed nucleases to cut deoxyribonucleic acid (DNA) at precise target locations in the genome, thereby creating double-strand or single-strand DNA breaks at particular locations within the genome. Such breaks can be and regularly are repaired by natural, endogenous cellular processes, such as homology-directed repair (HDR) and non-homologous end-joining (NHEJ), as recently reviewed in Cox et al., Nature Medicine 21(2), 121-31 (2015). NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence may be in the endogenous genome, such as a sister chromatid. Alternatively, the donor may be an exogenous nucleic acid, such as a plasmid, a single-strand oligonucleotide, a double-stranded oligonucleotide, a duplex oligonucleotide or a virus, that has regions of high homology with the nuclease-cleaved locus, but which may also contain additional sequence or sequence changes including deletions that can be incorporated into the cleaved target locus. A third repair mechanism is microhomology-mediated end joining (MMEJ), also referred to as “Alternative NHEJ”, in which the genetic outcome is similar to NHEJ in that small deletions and insertions can occur at the cleavage site. MMEJ makes use of homologous sequences of a few basepairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi:10.1038/nsmb.2961(2015); Mateos-Gomez et al., Nature 518, 254-57 (2015); Ceccaldi et al., Nature 528, 258-62 (2015). In some instances it may be possible to predict likely repair outcomes based on analysis of potential microhomologies at the site of the DNA break.

Each of these genome editing mechanisms can be used to create desired genomic alterations. A step in the genome editing process is to create one or two DNA breaks, the latter as double-strand breaks or as two single-stranded breaks, in the target locus as close as possible to the site of intended mutation. This can be achieved via the use of site-directed polypeptides, as described and illustrated herein.

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair or non-homologous end joining or alternative non-homologous end joining (A-NHEJ) or microhomology-mediated end joining). NHEJ can repair cleaved target nucleic acid without the need for a homologous template. This can sometimes result in small deletions or insertions (indels) in the target nucleic acid at the site of cleavage, and can lead to disruption or alteration of gene expression.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the locus, biogenesis of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” The repeats can form hairpin structures and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. Some Cas genes comprise homologous secondary and/or tertiary structures.

Type II CRISPR Systems

crRNA biogenesis in a Type II CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed polypeptide (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096):816-821 (2012) showed that the CRISPR/Cas9 system is useful for RNA-programmable genome editing, and international patent application publication number WO2013/176772 provides numerous examples and applications of the CRISPR/Cas endonuclease system for site-specific gene editing.

Type V CRISPR Systems

Type V CRISPR systems have several important differences from Type II systems. For example, Cpf1 is a single RNA-guided endonuclease that, in contrast to Type II systems, lacks tracrRNA. In fact, Cpf1-associated CRISPR arrays are processed into mature crRNAS without the requirement of an additional trans-activating tracrRNA. The Type V CRISPR array is processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems start with 20-24 nucleotides of spacer sequence followed by about 22 nucleotides of direct repeat. Also, Cpf1 utilizes a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

Cas Genes/Polypeptides and Protospacer Adjacent Motifs

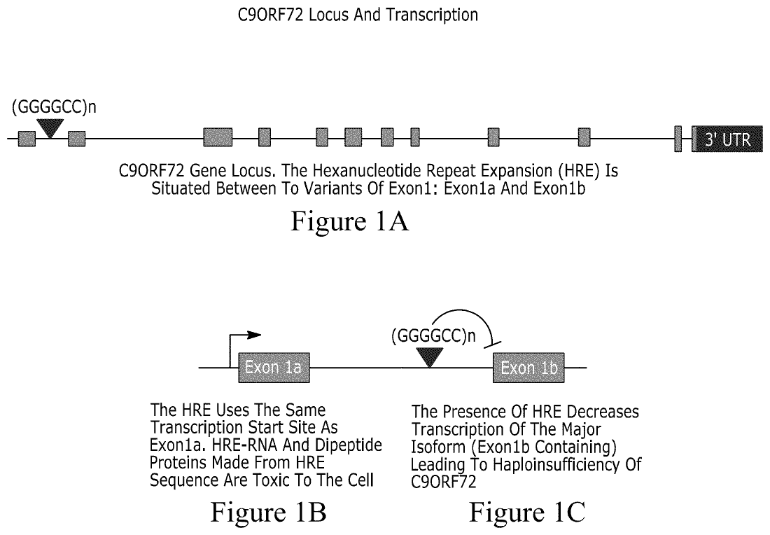

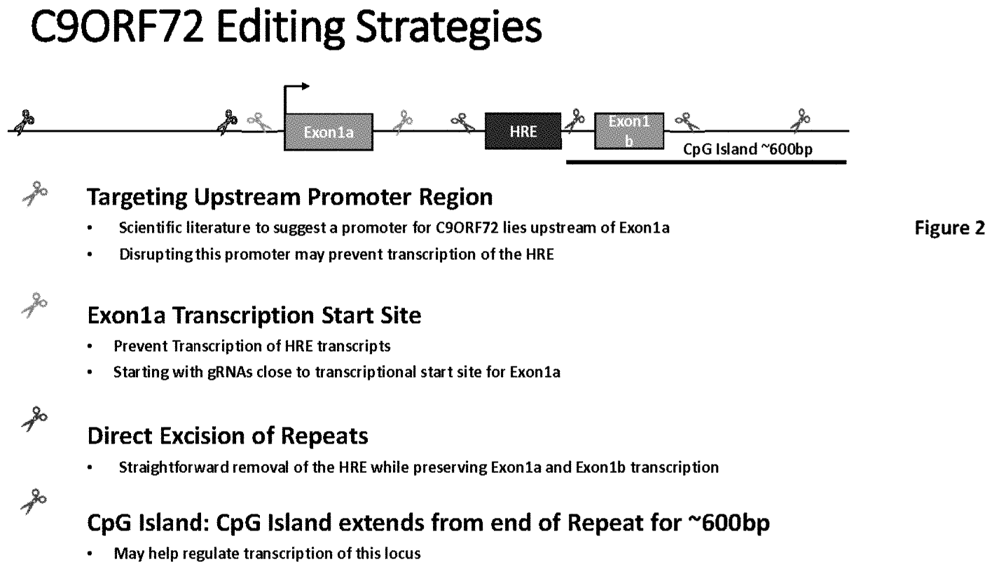

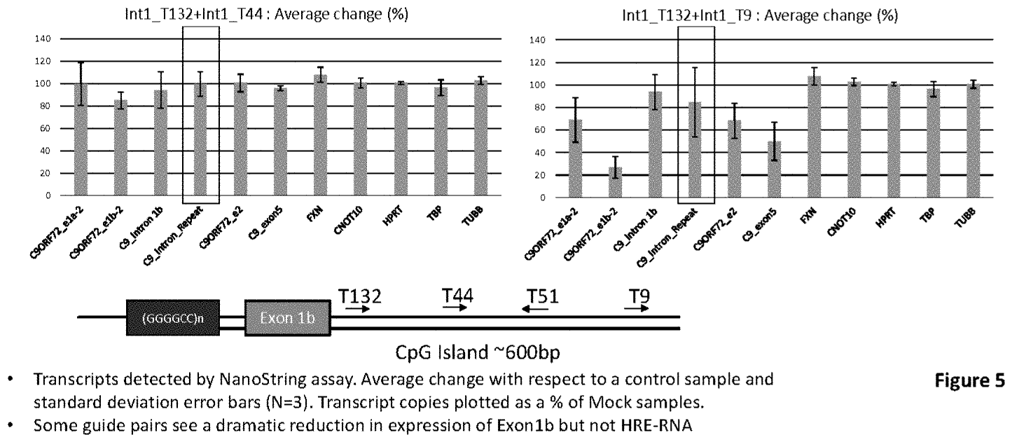

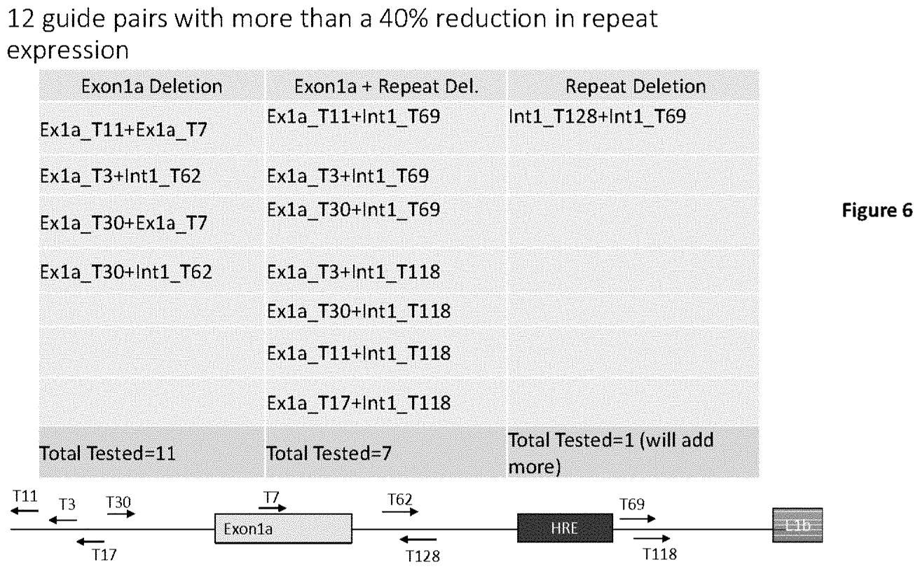

Exemplary CRISPR/Cas polypeptides include the Cas9 polypeptides in FIG. 1 of Fonfara et al., Nucleic Acids Research, 42: 2577-2590 (2014). The CRISPR/Cas gene naming system has undergone extensive rewriting since the Cas genes were discovered. FIG. 5 of Fonfara, supra, provides PAM sequences for the Cas9 polypeptides from various species.

Site-Directed DNA Endonucleases

A site-directed endonuclease is a nuclease used in genome editing to cleave DNA. The site-directed endonuclease may be administered to a cell or a patient as either: one or more polypeptides, or one or more mRNAs encoding the polypeptide.

In the context of a CRISPR/Cas or CRISPR/Cpf1 system, the site-directed DNA endonuclease can bind to a guide RNA that, in turn, specifies the site in the target DNA to which the polypeptide is directed.

In some embodiments, a DNA endonuclease comprises a plurality of nucleic acid-cleaving (i.e., nuclease) domains. Two or more nucleic acid-cleaving domains can be linked together via a linker. In some embodiments, the linker comprises a flexible linker. Linkers may comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

Naturally-occurring wild-type Cas9 enzymes comprise two nuclease domains, a HNH nuclease domain and a RuvC domain. Herein, the “Cas9” refers to both naturally-occurring and recombinant Cas9s. Cas9 enzymes contemplated herein comprises a HNH or HNH-like nuclease domain, and/or a RuvC or RuvC-like nuclease domain.

HNH or HNH-like domains comprise a McrA-like fold. HNH or HNH-like domains comprises two antiparallel β-strands and an α-helix. HNH or HNH-like domains comprises a metal binding site (e.g., a divalent cation binding site). HNH or HNH-like domains can cleave one strand of a target nucleic acid (e.g., the complementary strand of the crRNA targeted strand).

RuvC or RuvC-like domains comprise an RNaseH or RNaseH-like fold. RuvC/RNaseH domains are involved in a diverse set of nucleic acid-based functions including acting on both RNA and DNA. The RNaseH domain comprises 5 β-strands surrounded by a plurality of α-helices. RuvC/RNaseH or RuvC/RNaseH-like domains comprise a metal binding site (e.g., a divalent cation binding site). RuvC/RNaseH or RuvC/RNaseH-like domains can cleave one strand of a target nucleic acid (e.g., the non-complementary strand of a double-stranded target DNA).

DNA endonucleases can introduce double-strand breaks (or single-strand breaks) in nucleic acids, e.g., genomic DNA. The double-strand break can stimulate a cell's endogenous DNA-repair pathways (e.g., homology-dependent repair (HDR) or non-homologous end joining (NHEJ) or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage, and can lead to disruption or alteration of gene expression. In some embodiments, the DNA endonuclease comprises a nucleotide sequence that encodes an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US2014/0068797 Sequence ID No. 8 or Sapranauskas et al., Nucleic Acids Res, 39(21): 9275-9282 (2011)], and various other site-directed polypeptides).

In some embodiments, the DNA endonuclease comprises a nucleotide sequence that encodes an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes, supra).

In some embodiments, the DNA endonuclease comprises a nucleotide sequence that encodes an amino acid sequence at least 70, 75, 80, 85, 90, 95, 97, 99, or 100% identity to a wild-type site-directed polypeptide (e.g., Cas9 from S. pyogenes, supra) over 10 contiguous amino acids. In some embodiments, the DNA endonuclease comprises a nucleotide sequence that encodes an amino acid sequence comprises at most: 70, 75, 80, 85, 90, 95, 97, 99, or 100% identity to a wild-type site-directed polypeptide (e.g., Cas9 from S. pyogenes, supra) over 10 contiguous amino acids. In some embodiments, the DNA endonuclease comprises a nucleotide sequence that encodes an amino acid sequence at least: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the encoded site-directed polypeptide. In some embodiments, the DNA endonuclease comprises a nucleotide sequence that encodes an amino acid sequence at most: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the encoded site-directed polypeptide. In some embodiments, the DNA endonuclease comprises a nucleotide sequence that encodes an amino acid sequence at least: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the encoded site-directed polypeptide. In some embodiments, the DNA endonuclease comprises a nucleotide sequence that encodes an amino acid sequence at most: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the encoded site-directed polypeptide.

In some embodiments, the DNA endonuclease encodes a site-directed polypeptide comprising a modified form of a wild-type exemplary site-directed polypeptide. The modified form of the wild-type exemplary site-directed polypeptide comprises a mutation that reduces the nucleic acid-cleaving activity of the site-directed polypeptide. In some embodiments, the modified form of the wild-type exemplary site-directed polypeptide has less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

In some embodiments, the site-directed polypeptide comprises an amino acid sequence comprising at least 15% amino acid identity to a Cas9 from a bacterium (e.g., S. pyogenes), a nucleic acid binding domain, and two nucleic acid cleaving domains (i.e., a HNH domain and a RuvC domain).

In some embodiments, the site-directed polypeptide comprises an amino acid sequence comprising at least 15% amino acid identity to a Cas9 from a bacterium (e.g., S. pyogenes), and two nucleic acid cleaving domains (i.e., a HNH domain and a RuvC domain).

In some embodiments, the site-directed polypeptide comprises an amino acid sequence comprising at least 15% amino acid identity to a Cas9 from a bacterium (e.g., S. pyogenes), and two nucleic acid cleaving domains, wherein one or both of the nucleic acid cleaving domains comprise at least 50% amino acid identity to a nuclease domain from Cas9 from a bacterium (e.g., S. pyogenes).

In some embodiments, the site-directed polypeptide comprises an amino acid sequence comprising at least 15% amino acid identity to a Cas9 from a bacterium (e.g., S. pyogenes), two nucleic acid cleaving domains (i.e., a HNH domain and a RuvC domain), and non-native sequence (for example, a nuclear localization signal) or a linker linking the site-directed polypeptide to a non-native sequence.

In some embodiments, the site-directed polypeptide comprises an amino acid sequence comprising at least 15% amino acid identity to a Cas9 from a bacterium (e.g., S. pyogenes), two nucleic acid cleaving domains (i.e., a HNH domain and a RuvC domain), wherein the site-directed polypeptide comprises a mutation in one or both of the nucleic acid cleaving domains that reduces the cleaving activity of the nuclease domains by at least 50%.

In some embodiments, the site-directed polypeptide comprises an amino acid sequence comprising at least 15% amino acid identity to a Cas9 from a bacterium (e.g., S. pyogenes), and two nucleic acid cleaving domains (i.e., a HNH domain and a RuvC domain), wherein one of the nuclease domains comprises mutation of aspartic acid 10, and/or wherein one of the nuclease domains comprises mutation of histidine 840, and wherein the mutation reduces the cleaving activity of the nuclease domain(s) by at least 50%.

In some embodiments, the site-directed polypeptide (Cas9 protein) is from S. lugdunensis (SluCas9). In some embodiments, the Cas9 protein are from Staphylococcus aureus (SaCas9). In some embodiments, a suitable Cas9 protein for use in the present disclosure is any disclosed in WO2019/183150 and WO2019/118935, each of which is incorporate herein by reference.

In some embodiments of the invention, the one or more site-directed polypeptides, e.g. DNA endonucleases, include two nickases that together effect one double-strand break at a specific locus in the genome, or four nickases that together effect two double-strand breaks at specific loci in the genome. Alternatively, one site-directed polypeptide, e.g. DNA endonuclease, effects one double-strand break at a specific locus in the genome.

A Type-II CRISPR/Cas system component are from a Type-IIA, Type-IIB, or Type-IIC system. Cas9 and its orthologs are encompassed. Non-limiting exemplary species that the Cas9 nuclease or other components are from include Streptococcus pyogenes, Streptoccoccus lugdunensis, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein are from Streptococcus pyogenes (SpCas9). In some embodiments, the Cas9 protein is from S. lugdunensis (SluCas9). In some embodiments, the Cas9 protein are from Staphylococcus aureus (SaCas9). In some embodiments, a suitable Cas9 protein for use in the present disclosure is any disclosed in WO2019/183150 and WO2019/118935, each of which is incorporate herein by reference.

Guide RNAs

A guide RNA (or ‘gRNA”) comprises at least a spacer sequence that hybridizes to a target nucleic acid sequence of interest, and a CRISPR repeat sequence. In Type II systems, the gRNA also comprises a tracrRNA sequence. In the Type II guide RNA, the CRISPR repeat sequence and tracrRNA sequence hybridize to each other to form a duplex. In the Type V guide RNA, the crRNA forms a duplex. In both systems, the duplex binds a site-directed polypeptide, such that the guide RNA and site-direct polypeptide form a complex. The guide RNA provides target specificity to the complex by virtue of its association with the site-directed polypeptide. The guide RNA thus directs the activity of the site-directed polypeptide.

In some embodiments, the guide RNA is double-stranded. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

In some embodiments, the guide RNA is single-stranded guide. A single-molecule guide RNA in a Type II system comprises, in the 5′ to 3′ direction, an optional spacer extension sequence, a spacer sequence, a minimum CRISPR repeat sequence, a single-stranded guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-stranded guide linker links the minimum CRISPR repeat and the minimum tracrRNA sequence to form a hairpin structure. The optional tracrRNA extension comprises one or more hairpins.

A single-stranded guide RNA in a Type V system comprises, in the 5′ to 3′ direction, a minimum CRISPR repeat sequence and a spacer sequence.

By way of illustration, guide RNAs used in the CRISPR/Cas system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some embodiments, the one or more gRNAs comprises a nucleotide sequence set forth in SEQ ID NOs: 1-9. In some embodiments, the two gRNAs are set forth in (a) SEQ ID NOs: 1 and 2 (T11 and T7); (b) SEQ ID NOs: 3 and 4 (T3 and T62); (c) SEQ ID NOs: 5 and 2 (T30 and T7); (d) SEQ ID NOs: 5 and 4 (T30 and T62); (e) SEQ ID NOs: 1 and 6 (T11 and T69); (f) SEQ ID NOs: 3 and 6 (T3 and T69); (g) SEQ ID NOs: 5 and 6 (T30 and T69); (h) SEQ ID NOs: 3 and 7 (T3 and T118); (i) SEQ ID NOs: 5 and 7 (T30 and T118); (j) SEQ ID NOs: 1 and 8 (T11 and T118); (k) SEQ ID NOs: 8 and 7 (T17 and T118); or (l) SEQ ID NOs: 9 and 6 (T128 and T69). In some embodiments, the two gRNAs are set forth in (a) SEQ ID NOs: 1 and 2 (T11 and T7); (b) SEQ ID NOs: 3 and 4 (T3 and T62); (c) SEQ ID NOs: 5 and 2 (T30 and T7); or (d) SEQ ID NOs: 5 and 4 (T30 and T62). In some embodiments, the two gRNAs are set forth in (a) SEQ ID NOs: 1 and 6 (T11 and T69); (b) SEQ ID NOs: 3 and 6 (T3 and T69); (c) SEQ ID NOs: 5 and 6 (T30 and T69); (d) SEQ ID NOs: 3 and 7 (T3 and T118); (e) SEQ ID NOs: 5 and 7 (T30 and T118); (f) SEQ ID NOs: 1 and 7 (T11 and T118); or (g) SEQ ID NOs: 8 and 7 (T17 and T118). In some embodiments, the two gRNAs are SEQ ID NO: 9 and SEQ ID NO: 6 (T128 and T69). In some embodiments, the two gRNAs are SEQ ID NO: 1 and SEQ ID NO: 4 (T11 and T62).

In some embodiments, the one or more gRNAs comprises a nucleotide sequence set forth in SEQ ID NOs: 17-41. In some embodiments, the one or more gRNAs are SEQ ID NO: 17 and SEQ ID NO: 18 (S2 and S26). In some embodiment, the one or more gRNAs are SEQ ID NO: 17 and SEQ ID NO: 20 (S3 and S20). In some embodiments, the one or more gRNAs are SEQ ID NO: 20 and SEQ ID NO: 21 (S2 and S24). In some embodiments, the one or more gRNAs are SEQ ID NO: 20 and SEQ ID NO: 22 (S3 and S31). In some embodiments, the one or more gRNAs are SEQ ID NO: 23 and SEQ ID NO: 24 (S15 and S22). In some embodiments, the one or more gRNAs are SEQ ID NO: 25 and SEQ ID NO: 24 (S14 and S22). In some embodiments, the one or more gRNAs are SEQ ID NO: 26 and SEQ ID NO: 18 (S17 and S26). In some embodiments, the one or more gRNAs are SEQ ID NO: 26 and SEQ ID NO: 19 (S17 and S20). In some embodiments, the one or more gRNAs are SEQ ID NO: 27 and SEQ ID NO; 28 (S16 and S30). In some embodiments, the one or more gRNAs are SEQ ID NO: 29 and SEQ ID NO: 22 (S32 and S31). In some embodiments, the one or more gRNAs are SEQ ID NO: 31 and SEQ ID NO: 40 (S28 and S29). In some embodiments, the one or more gRNAs are SEQ ID NO: 41 and SEQ ID NO: 24 (51 and S22). In some embodiments, the one or more gRNAs are SEQ ID NO: 20 and SEQ ID NO: 34 (S2 and S9). In some embodiments, the one or more gRNAs are SEQ ID NO: 17 and SEQ ID NO: 32 (S3 and S5). In some embodiments, the one or more gRNAs are SEQ ID NO: 17 and SEQ ID NO: 33 (S3 and S6). In some embodiments, the one or more gRNAs are SEQ ID NO: 17 and SEQ ID NO: 34 (S3 and S9).

In some embodiments, the one or more gRNAs are (a) SEQ ID NO: 1 and SEQ ID NO: 2 (T1 and T7); (b) SEQ ID NO: 1 and SEQ ID NO: 7 (T1 and T118); (c) SEQ ID NO: 1 and SEQ ID NO: 6 (T1 and T69); (d) SEQ ID NO: 8 and SEQ ID NO: 7 (T17 and T118; (e) SEQ ID NO: 1 and SEQ ID NO: 15 (T1 and T5); (f) SEQ ID NO: 3 and SEQ ID NO: 7 (T3 and T118); (g) SEQ ID NO: 3 and SEQ ID NO: 15 (T3 and T5); (h) SEQ ID NO: 3 and SEQ ID NO: 6 (T3 and T69); (i) SEQ ID NO: 5 and SEQ ID NO: 2 (T30 and T7); (j) SEQ ID NO: 5 and SEQ ID NO: 7 (T30 and T118); (k) SEQ ID NO: 5 and SEQ ID NO: 15 (T30 and T5); (l) SEQ ID NO: 5 and SEQ ID NO: 6 (T30 and T69); (m) SEQ ID NO: 9 and SEQ ID NO: 6 (T128 and T69); or (n) SEQ ID NO: 5 and SEQ ID NO: 4 (T30 and T62).

In some embodiments, the one or more gRNAs are (a) SEQ ID NO: 20 and SEQ ID NO: 21 (S2 and S24); (b) SEQ ID NO: 20 and SEQ ID NO: 22 (S2 and S31); (c) SEQ ID NO: 26 and SEQ ID NO: 18 (S17 and S26); (d) SEQ ID NO: 26 and SEQ ID NO: 29 (S28 and S29); (e) SEQ ID NO: 41 and SEQ ID NO: 24 (51 and S22); (f) SEQ ID NO: 20 and SEQ ID NO: 34 (S2 and S9); (g) SEQ ID NO: 17 and SEQ ID NO: 33 (S3 and S6); or (h) SEQ ID NO: 20 and SEQ ID NO: 33 (S2 and S6).

In some embodiments, the one or more gRNAs are (a) SEQ ID NO: 6 and SEQ ID NO: 21 (S2 and S24), (b) SEQ ID NO: 6 and SEQ ID NO: 22 (S2 and S31), (c) SEQ ID NO: 6 and SEQ ID NO: 33 (S2 and S6), (d) SEQ ID NO: 6 and SEQ ID NO: 34 (S2 and S9), (e) SEQ ID NO: 17 and SEQ ID NO: 33 (S3 and S6), (f) SEQ ID NO: 26 and SEQ ID NO: 18 (S17 and S26), or (g) SEQ ID NO: 31 and SEQ ID NO: 40 (S28 and S29).

Nucleic Acid Modifications

In certain embodiments, modified polynucleotides are used in the CRISPR/Cas9/Cpf1 system, in which case the guide RNAs (either single-molecule guides or double-molecule guides) and/or a DNA or an RNA encoding a Cas or Cpf1 endonuclease introduced into a cell can be modified. Such modified polynucleotides can be used in the CRISPR/Cas9/Cpf1 system to edit any one or more genomic loci.

Modified guide RNAs can be used to enhance the formation or stability of the CRISPR/Cas9/Cpf1 genome editing complex comprising guide RNAs, which may be single-molecule guides or double-molecule, and a Cas or Cpf1 endonuclease. Modifications of guide RNAs can also or alternatively be used to enhance the initiation, stability or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas or Cpf1 endonuclease is introduced into the cell to be edited via an RNA that needs to be translated in order to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (i.e. the endonuclease), and/or modifications that decrease the likelihood or degree to which the RNAs introduced into cells elicit innate immune responses.

Combinations of modifications can likewise be used. In the case of CRISPR/Cas9/Cpf1, for example, one or more types of modifications can be made to guide RNAs, and/or one or more types of modifications can be made to RNAs encoding Cas or Cpf1 endonuclease.

By way of illustration, guide RNAs used in the CRISPR/Cas9/Cpf1 system, or other smaller RNAs can be readily synthesized by chemical means, enabling a number of modifications to be readily incorporated. One approach used for generating chemically-modified RNAs of greater length is to produce two or more molecules that are ligated together. Much longer RNAs, such as those encoding a Cas9 endonuclease, are more readily generated enzymatically. While fewer types of modifications are generally available for use in enzymatically produced RNAs, there are still modifications that can be used to, e.g., enhance stability, reduce the likelihood or degree of innate immune response, and/or enhance other attributes.

By way of illustration of various types of modifications, especially those used frequently with smaller chemically synthesized RNAs, modifications can comprise one or more nucleotides modified at the 2′ position of the sugar, in some embodiments a 2′-O-alkyl, 2′-O-alkyl-O-alkyl, or 2′-fluoro-modified nucleotide. In some embodiments, RNA modifications include 2′-fluoro, 2′-amino or 2′ O-methyl modifications on the ribose of pyrimidines, abasic residues, or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones [see De Mesmaeker et al., Ace. Chem. Res., 28:366-374 (1995)]; morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, Biochemistry, 41(14): 4503-4510 (2002); Genesis, Volume 30, Issue 3, (2001); Heasman, Dev. Biol., 243: 209-214 (2002); Nasevicius et al., Nat. Genet., 26:216-220 (2000); Lacerra et al., Proc. Natl. Acad. Sci., 97: 9591-9596 (2000); and U.S. Pat. No. 5,034,506, issued Jul. 23, 1991.

Cyclohexenyl nucleic acid oligonucleotide mimetics are described in Wang et al., J. Am. Chem. Soc., 122: 8595-8602 (2000).

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)n CH₃, O(CH₂)ₙNH₂, or O(CH₂)ₙCH₃, where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some embodiments, a modification includes 2′-methoxyethoxy (2′-O—CH₂CH₂OCH₃, also known as 2′-O-(2-methoxyethyl)) (Martin et al, Helv. Chim. Acta, 1995, 78, 486). Other modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics, such as cyclobutyls in place of the pentofuranosyl group.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262. Further teaching of PNA compounds can be found in Nielsen et al, Science, 254: 1497-1500 (1991).

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Kornberg, A., DNA Replication, W. H. Freeman & Co., San Francisco, pp 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are embodiments of base substitutions.

Modified nucleobases comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine, and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, ‘Antisense Research and Applications’, CRC Press, Boca Raton, 1993, pp. 276-278) and are embodiments of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and U.S. Patent Application Publication 2003/0158403.

Thus, the term “modified” refers to a non-natural sugar, phosphate, or base that is incorporated into a guide RNA, an endonuclease, or both a guide RNA and an endonuclease. It is not necessary for all positions in a given oligonucleotide to be uniformly modified, and in fact more than one of the aforementioned modifications may be incorporated in a single oligonucleotide, or even in a single nucleoside within an oligonucleotide.

In some embodiments, the guide RNAs and/or mRNA (or DNA) encoding an endonuclease are chemically linked to one or more moieties or conjugates that enhance the activity, cellular distribution, or cellular uptake of the oligonucleotide. Such moieties comprise, but are not limited to, lipid moieties such as a cholesterol moiety [Letsinger et al., Proc. Natl. Acad. Sci. USA, 86: 6553-6556 (1989)]; cholic acid [Manoharan et al., Bioorg. Med. Chem. Let., 4: 1053-1060 (1994)]; a thioether, e.g., hexyl-S-tritylthiol [Manoharan et al, Ann. N. Y. Acad. Sci., 660: 306-309 (1992) and Manoharan et al., Bioorg. Med. Chem. Let., 3: 2765-2770 (1993)]; a thiocholesterol [Oberhauser et al., Nucl. Acids Res., 20: 533-538 (1992)]; an aliphatic chain, e.g., dodecandiol or undecyl residues [Kabanov et al., FEBS Lett., 259: 327-330 (1990) and Svinarchuk et al., Biochimie, 75: 49-54 (1993)]; a phospholipid, e.g., di-hexadecyl-rac-glycerol or triethylammonium 1,2-di-O-hexadecyl-rac-glycero-3-H-phosphonate [Manoharan et al., Tetrahedron Lett., 36: 3651-3654 (1995) and Shea et al., Nucl. Acids Res., 18: 3777-3783 (1990)]; a polyamine or a polyethylene glycol chain [Mancharan et al., Nucleosides & Nucleotides, 14: 969-973 (1995)]; adamantane acetic acid [Manoharan et al., Tetrahedron Lett., 36: 3651-3654 (1995)]; a palmityl moiety [(Mishra et al., Biochim. Biophys. Acta, 1264: 229-237 (1995)]; or an octadecylamine or hexylamino-carbonyl-t oxycholesterol moiety [Crooke et al., J. Pharmacol. Exp. Ther., 277: 923-937 (1996)]. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., Protein Pept Lett. 21(10):1025-30 (2014). Other systems known in the art and regularly developed can be used to target biomolecules of use in the present case and/or complexes thereof to particular target cells of interest.

These targeting moieties or conjugates can include conjugate groups covalently bound to functional groups, such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Longer polynucleotides that are less amenable to chemical synthesis and are typically produced by enzymatic synthesis can also be modified by various means. Such modifications can include, for example, the introduction of certain nucleotide analogs, the incorporation of particular sequences or other moieties at the 5′ or 3′ ends of molecules, and other modifications. By way of illustration, the mRNA encoding Cas9 is approximately 4 kb in length and can be synthesized by in vitro transcription. Modifications to the mRNA can be applied to, e.g., increase its translation or stability (such as by increasing its resistance to degradation with a cell), or to reduce the tendency of the RNA to elicit an innate immune response that is often observed in cells following introduction of exogenous RNAs, particularly longer RNAs such as that encoding Cas9.

Numerous such modifications have been described in the art, such as polyA tails, 5′ cap analogs (e.g., Anti Reverse Cap Analog (ARCA) or m7G(5′)ppp(5′)G (mCAP)), modified 5′ or 3′ untranslated regions (UTRs), use of modified bases (such as Pseudo-UTP, 2-Thio-UTP, 5-Methylcytidine-5′-Triphosphate (5-Methyl-CTP) or N6-Methyl-ATP), or treatment with phosphatase to remove 5′ terminal phosphates. These and other modifications are known in the art, and new modifications of RNAs are regularly being developed.

There are numerous commercial suppliers of modified RNAs, including for example, TriLink Biotech, AxoLabs, Bio-Synthesis Inc., Dharmacon and many others. As described by TriLink, for example, 5-Methyl-CTP can be used to impart desirable characteristics, such as increased nuclease stability, increased translation or reduced interaction of innate immune receptors with in vitro transcribed RNA. 5-Methylcytidine-5′-Triphosphate (5-Methyl-CTP), N6-Methyl-ATP, as well as Pseudo-UTP and 2-Thio-UTP, have also been shown to reduce innate immune stimulation in culture and in vivo while enhancing translation, as illustrated in publications by Kormann et al. and Warren et al. referred to below.

It has been shown that chemically modified mRNA delivered in vivo can be used to achieve improved therapeutic effects; see, e.g., Kormann et al., Nature Biotechnology 29, 154-157 (2011). Such modifications can be used, for example, to increase the stability of the RNA molecule and/or reduce its immunogenicity. Using chemical modifications such as Pseudo-U, N6-Methyl-A, 2-Thio-U and 5-Methyl-C, it was found that substituting just one quarter of the uridine and cytidine residues with 2-Thio-U and 5-Methyl-C respectively resulted in a significant decrease in toll-like receptor (TLR) mediated recognition of the mRNA in mice. By reducing the activation of the innate immune system, these modifications can be used to effectively increase the stability and longevity of the mRNA in vivo; see, e.g., Kormann et al., supra.

It has also been shown that repeated administration of synthetic messenger RNAs incorporating modifications designed to bypass innate anti-viral responses can reprogram differentiated human cells to pluripotency. See, e.g., Warren, et al., Cell Stem Cell, 7(5):618-30 (2010). Such modified mRNAs that act as primary reprogramming proteins can be an efficient means of reprogramming multiple human cell types. Such cells are referred to as induced pluripotency stem cells (iPSCs), and it was found that enzymatically synthesized RNA incorporating 5-Methyl-CTP, Pseudo-UTP and an Anti Reverse Cap Analog (ARCA) could be used to effectively evade the cell's antiviral response; see, e.g., Warren et al., supra.

Other modifications of polynucleotides described in the art include, for example, the use of polyA tails, the addition of 5′ cap analogs (such as m7G(5′)ppp(5′)G (mCAP)), modifications of 5′ or 3′ untranslated regions (UTRs), or treatment with phosphatase to remove 5′ terminal phosphates—and new approaches are regularly being developed.

Finally, a number of conjugates can be applied to polynucleotides, such as RNAs, for use herein that can enhance their delivery and/or uptake by cells, including for example, cholesterol, tocopherol and folic acid, lipids, peptides, polymers, linkers and aptamers; see, e.g., the review by Winkler, Ther. Deliv. 4:791-809 (2013), and references cited therein.

Target Nucleic Acid Sequence

The guide RNA hybridizes to a target nucleic acid sequence upstream or within the C9ORF72 gene. In some embodiments, the target nucleic acid sequence comprises 20 nucleotides in length. In some embodiments, the target nucleic acid comprises more than 20 nucleotides in length. In some embodiments, the target nucleic acid comprises less than 20 nucleotides in length. In some embodiments, the target nucleic acid comprises at least: 5, 10, 15, 16, 17, 18, 19, 20, 21, 22, 23, 24, 25, 30 or more nucleotides in length. In some embodiments, the target nucleic acid comprises at most: 5, 10, 15, 16, 17, 18, 19, 20, 21, 22, 23, 24, 25, 30 or more nucleotides in length.

In some embodiments, the target sequence is within region of the C9ORF72 gene comprising nucleotides 1801-2900 of SEQ ID NO: 42. In some embodiments, the target sequence is within a region of the C9ORF72 gene comprises nucleotides 1801-1970 of SEQ ID NO: 42. In some embodiments, the target sequence is within a region of the C9ORF72 gene comprising nucleotides 2051-2156 of SEQ ID NO: 42. In some embodiments, the target sequence is within a region of the C9ORF72 gene comprising nucleotides 2189-2326 of SEQ ID NO: 42. In some embodiments, the target sequence is within a region of the C9ORF72 gene comprising nucleotides 2384-2900 of SEQ ID NO: 42.

In some embodiments, the target sequence is within a region of the C9ORF72 gene comprising nucleotides 1801-1970 of SEQ ID NO: 42 and nucleotides 2051-2156 of SEQ ID NO: 42.

In some embodiments, the target sequence is within a region of the C9ORF72 gene comprising nucleotides 1801-1970 of SEQ ID NO: 42 and nucleotides 2189-2326 of SEQ ID NO: 42.

In some embodiments, the target sequence is within a region of the C9ORF72 gene comprising nucleotides 1801-1970 of SEQ ID NO: 42 and nucleotides 2384-2900 of SEQ ID NO: 42.

Therapeutic Methods

ALS patients exhibit an expanded hexanucleotide repeat in the C9ORF72 gene. Therefore, different patients will generally require similar correction strategies. Any CRISPR DNA endonuclease may be used in the methods described herein, each CRISPR endonuclease having its own associated PAM, which may or may not be disease specific. For example, g RNA spacer sequences for targeting the C9ORF72 gene with a CRISPR/Cas9 endonuclease from S. pyogenes, S. aureus, S. thermophiles, T. denticola, N. meningitides, Acidominococcus and Lachnospiraceae have been identified in International Publication No. WO 2017/109757, the disclosure of which is incorporated herein by reference in its entirety.

In some embodiments, the one or more DSBs are upstream of the transcription start site of exon1a. In some embodiments, the one or more DSBs are within an upstream sequence region of the C9ORF72 gene. As used herein, the term “upstream sequence” means a region upstream of the first nucleotide of exon 1a and optionally including promoter sequences, transcription start site sequences, and thus includes a region stretching 10, 15, 20, 25, 30, 35, 40, 45, 50, 55, 60, 65, 70, 75, 80, 85, 90, 95, 100, 110, 120, 125, 130, 135, 140, 145, 150, 155, 160, 165, 170, 175, 180, 185, 190, 195, 200, 250, 300, 350, 400, 450, 500 or more nucleotide upstream of exon 1a. In some embodiments, the one or more DSBs are within 500 nucleotides of the transcription start site for exon1a. In some embodiments, the one or more DSBs are within at least 200 nucleotides of the transcription start site for exon1a. In some embodiments, the one or more DSBs are within at least 10, 15, 20, 25, 30, 35, 40, 45, 50, 55, 60, 65, 70, 75, 80, 85, 90, 95, 100, 110, 120, 125, 130, 135, 140, 145, 150, 155, 160, 165, 170, 175, 180, 185, 190, 195, 200, 250, 300, 350, 400, 450 or 500 nucleotides of the transcriptional start site for exon1a.

In some embodiments, a single DSB is targeting the transcription start site of exon1a. The transcription start site of exon1a is located at Chromosome 9 and upstream of nucleotide 27,573,709 (Genome Reference Consortium—GRCh38/hg38). Exon1a is located at Chromosome 9 at nucleotides 27,573,709-27,573,866 (Genome Reference Consortium—GRCh38/hg38).

In some embodiments, a first DSB is upstream of the transcription start site of exon1a and a second DSB is in exon1a downstream of the transcription start site of exon1a. In some embodiments, the first DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) upstream of the transcription start site for exon1a. In some embodiments, the second DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) downstream of the transcriptional start site for exon1a.

In some embodiments, a first DSB is upstream of the transcription start site of exon1a and a second DSB is in intron 1 and upstream of the expanded hexanucleotide repeat. Intron 1 is located at chromosome 9 at nucleotides 27,567,165-27,573,708 (Genome Reference Consortium—GRCh38/hg38). In some embodiments, the first DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) upstream of the transcription start site for exon1a. In some embodiments, the second DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) upstream of the expanded hexanucleotide repeat.

In some embodiments, a first DSB is upstream of the transcription start site of exon1a and a second DSB is in intron 1 and within of the expanded hexanucleotide repeat. In some embodiments, the first DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) upstream of the transcription start site for exon1a. In some embodiments, the second DSB is within the first 5-10 nucleotides (e.g., 5, 6, 7, 8, 9, 10 nucleotides) of the expanded hexanucleotide repeat. In some embodiments, the second DSB is within the last 5-10 nucleotides (e.g., 5, 6, 7, 8, 9, 10 nucleotides) of the expanded hexanucleotide repeat.

In some embodiments, a first DSB is upstream of the transcription start site of exon1a and a second DSB is in intron 1 and downstream of the hexanucleotide repeat. The hexanucleotide repeat is located at Chromosome 9 at nucleotides 27,573,529-27,573,546 (Genome Reference Consortium—GRCh38/hg38). In some embodiments, the first DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) upstream of the transcription start site for exon1a. In some embodiments, the second DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) downstream of the expanded hexanucleotide repeat.

In some embodiments, a first DSB is upstream of the transcription start site of exon1a and a second DSB is in intron 1 and downstream of the hexanucleotide repeat. The hexanucleotide repeat is located at Chromosome 9 at nucleotides 27,573,529-27,573,546 (Genome Reference Consortium—GRCh38/hg38). In some embodiments, the first DSB is at least 1 nucleotides (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) upstream of the expanded hexanucleotide repeat. In some embodiments, the second DSB is at least 1 nucleotide (e.g., at least 1, 2, 3, 4, 5, 6, 7, 8, 9, 10, 15, 20, 25, 30, 35, 40, 45, 50, 55, 60, 65, 70, 75, 80, 85, 90, 95, 100, 150, or 200 nucleotides) downstream of the expanded hexanucleotide repeat.

In some embodiments, a first DSB is upstream of the transcription start site of exon1a and a second DSB is in intron 1 and downstream of the hexanucleotide repeat. The hexanucleotide repeat is located at Chromosome 9 at nucleotides 27,573,529-27,573,546 (Genome Reference Consortium—GRCh38/hg38). In some embodiments, the second DSB is within the first 5-10 nucleotides (e.g., 5, 6, 7, 8, 9, 10 nucleotides) of the expanded hexanucleotide repeat. In some embodiments, the second DSB is within the last 5-10 nucleotides (e.g., 5, 6, 7, 8, 9, 10 nucleotides) of the expanded hexanucleotide repeat.

In some embodiments, a first DSB is within nucleotides 1801-1970 of SEQ ID NO: 42 and a second DSB is within nucleotides 2051-2156 of SEQ ID NO: 42. In some embodiments, a first DSB is within nucleotides 1801-1970 of SEQ ID NO: 42 and a second DSB is within nucleotides 2189-2326 of SEQ ID NO: 42. In some embodiments, a first DSB is within nucleotides 1801-1970 of SEQ ID NO: 42 and a second DSB is within nucleotides 2384-2900 of SEQ ID NO: 42.

The ends from a DNA break or ends from different breaks can be joined using the several nonhomologous repair pathways in which the DNA ends are joined with little or no base-pairing at the junction. In addition to canonical NHEJ, there are similar repair mechanisms, such as alt-NHEJ. If there are two breaks, the intervening segment can be deleted or inverted. NHEJ repair pathways can lead to insertions, deletions or mutations at the joints.

For any of the genome editing strategies, gene editing can be confirmed by sequencing or PCR analysis.

Nucleic Acids Encoding System Components

In another aspect, the present disclosure provides a nucleic acid comprising a nucleotide sequence encoding one or more guide RNAs, and a DNA endonuclease.

In some embodiments, the nucleic acid encoding one or more guide RNAs and a DNA endonuclease comprises a vector (e.g., a recombinant expression vector). The term “vector” refers to a nucleic acid molecule capable of transporting another nucleic acid to which it has been linked. One type of vector is a “plasmid”, which refers to a circular double-stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some embodiments, vectors are capable of directing the expression of nucleic acids to which they are operatively linked. Such vectors are referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors may be used so long as they are compatible with the host cell.

In some embodiments, a vector comprises one or more transcription and/or translation control elements. Depending on the host/vector system utilized, any of a number of suitable transcription and translation control elements, including constitutive and inducible promoters, transcription enhancer elements, transcription terminators, etc. may be used in the expression vector. In some embodiments, the vector is a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas or Cpf1 endonuclease, various promoters such as RNA polymerase III promoters, including for example U6 and H1, can be advantageous. Descriptions of and parameters for enhancing the use of such promoters are known in art, and additional information and approaches are regularly being described; see, e.g., Ma, H. et al., Molecular Therapy—Nucleic Acids 3, e161 (2014) doi:10.1038/mtna.2014.12.

The expression vector may also contain a ribosome binding site for translation initiation and a transcription terminator. The expression vector may also include appropriate sequences for amplifying expression. The expression vector may also include nucleotide sequences encoding non-native tags (e.g., histidine tag, hemagglutinin tag, green fluorescent protein, etc.) that are fused to the site-directed polypeptide, thus resulting in a fusion protein.

In some embodiments, a promoter is an inducible promoter (e.g., a heat shock promoter, tetracycline-regulated promoter, steroid-regulated promoter, metal-regulated promoter, estrogen receptor-regulated promoter, etc.). In some embodiments, a promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.).

In some embodiments, the nucleic acid encoding one or more guide RNAs and/or DNA endonuclease are packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. A variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Delivery

Guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) may be delivered by non-viral delivery vehicles known in the art, such as electroporation or lipid nanoparticles. In further alternative embodiments, the DNA endonuclease may be delivered as one or more polypeptides, either alone or pre-complexed with one or more guide RNAs.

Polynucleotides may be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other polynucleotides).

Polynucleotides, such as guide RNA, sgRNA, and mRNA encoding an endonuclease, may be delivered to a cell or a patient by a lipid nanoparticle (LNP).

A LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm.

LNPs may be made from cationic, anionic, or neutral lipids. Neutral lipids, such as the fusogenic phospholipid DOPE or the membrane component cholesterol, may be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses.

LNPs may also be comprised of hydrophobic lipids, hydrophilic lipids, or both hydrophobic and hydrophilic lipids.

Any lipid or combination of lipids that are known in the art may be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

The lipids may be combined in any number of molar ratios to produce a LNP. In addition, the polynucleotide(s) may be combined with lipid(s) in a wide range of molar ratios to produce a LNP.

As stated previously, the DNA endonuclease and guide RNA may each be administered separately to a cell or a patient. On the other hand, the DNA endonuclease may be pre-complexed with one or more guide RNAs. The pre-complexed material may then be administered to a cell or a patient. Such pre-complexed material is known as a ribonucleoprotein particle (RNP).

RNA is capable of forming specific interactions with RNA or DNA. While this property is exploited in many biological processes, it also comes with the risk of promiscuous interactions in a nucleic acid-rich cellular environment. One solution to this problem is the formation of ribonucleoprotein particles (RNPs), in which the RNA is pre-complexed with an endonuclease. Another benefit of the RNP is protection of the RNA from degradation.

The DNA endonuclease in the RNP may be modified or unmodified.

Likewise, the gRNA may be modified or unmodified. Numerous modifications are known in the art and may be used.

The DNA endonuclease and gRNA can be generally combined in a 1:1 molar ratio. However, a wide range of molar ratios may be used to produce a RNP.

In some embodiments, an AAV vector is used for delivery. Exemplary AAV serotypes include, but are not limited to, AAV-1, AAV-2, AAV-3, AAV-4, AAV-5, AAV-6, AAV-7, AAV-8, AAV-9, AAV-10, AAV-11, AAV-12, AAV-13 and AAV rh.74. See also Table 1.

TABLE 1

| AAV | Genbank |
|---|---|
| Serotype | Accession No. |
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

A method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In addition to adeno-associated viral vectors, other viral vectors may be used in the practice of the invention. Such viral vectors include, but are not limited to, lentivirus, alphavirus, enterovirus, pestivirus, baculovirus, herpesvirus, Epstein Barr virus, papovavirus, poxvirus, vaccinia virus, and herpes simplex virus.

Options are available to deliver the Cas9 nuclease as a DNA plasmid, as mRNA or as a protein. The guide RNA can be expressed from the same DNA, or can also be delivered as an RNA. The RNA can be chemically modified to alter or improve its half-life, or decrease the likelihood or degree of immune response. The endonuclease protein can be complexed with the gRNA prior to delivery. Viral vectors allow efficient delivery; split versions of Cas9 and smaller orthologs of Cas9 can be packaged in AAV, as can donors for HDR. A range of non-viral delivery methods also exist that can deliver each of these components, or non-viral and viral methods can be employed in tandem. For example, nano-particles can be used to deliver the protein and guide RNA, while AAV can be used to deliver a donor DNA.

Therapeutic Approach

Provided herein are methods for treating a patient with amyotrophic lateral sclerosis (ALS) using genome engineering tools to create permanent changes to the genome by (1) modification the transcription start site of exon1a to render the transcription start site non-functioning, (2) deletion of the transcription site of exon1a, (3) deletion of exon1a, or (4) deletion of the expanded hexanucleotide repeat within or near the C9ORF72 gene, or any combinations of (1)-(4), above. In some embodiments, such methods use endonucleases, such as CRISPR associated (Cas9, Cpf1 and the like) nucleases, to modify the transcription start site of exon1a to render the transcription start site non-functioning; delete the transcription site of exon1a; delete exon1a; or delete the expanded hexanucleotide repeat of the C9ORF72 gene, or any combinations thereof.

In one embodiment, a method of treating or ameliorating the symptoms of ALS is provided, comprising editing the C9ORF72 gene in a human cell by genome editing comprising introducing into the cell one or more deoxyribonucleic acid (DNA) endonucleases to effect one or more double-strand breaks (DSBs) within or near the first exon of the C9ORF72 gene that results in modification or deletion of exon1a transcription start site within the C9ORF72 gene, or deletion of a hexanucleotide repeat within the C9ORF72 gene.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by standard clinical techniques.

Administration & Efficacy

Guide RNAs of the invention are formulated with pharmaceutically acceptable excipients such as carriers, solvents, stabilizers, adjuvants, diluents, etc., depending upon the particular mode of administration and dosage form. Guide RNA compositions are generally formulated to achieve a physiologically compatible pH, and range from a pH of about 3 to a pH of about 11, about pH 3 to about pH 7, depending on the formulation and route of administration. In alternative embodiments, the pH is adjusted to a range from about pH 5.0 to about pH 8. In some embodiments, the compositions comprise a therapeutically effective amount of at least one compound as described herein, together with one or more pharmaceutically acceptable excipients. Optionally, the compositions comprise a combination of the compounds described herein, or may include a second active ingredient useful in the treatment or prevention of bacterial growth (for example and without limitation, anti-bacterial or anti-microbial agents), or may include a combination of reagents of the invention.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

The terms “individual”, “subject,” “host” and “patient” are used interchangeably herein and refer to any subject for whom diagnosis, treatment or therapy is desired. In some embodiments, the subject is a mammal. In some embodiments, the subject is a human being.

Deletion of the expanded hexanucleotide repeats in the C9ORF72 gene in cells of patients having ALS can be beneficial for ameliorating one or more symptoms of the disease, for increasing long-term survival, and/or for reducing side effects associated with other treatments.

“Administered” refers to the delivery of a composition described herein comprising the two guide ribonucleic acid (gRNAs) and the one or more DNA endonucleases (or a vector comprising a polynucleotide that encodes the gRNAs and the one or more DNA endonucleases) into a subject by a method or route that results in at least partial localization of the composition at a desired site. A composition can be administered by any appropriate route that results in effective treatment in the subject, i.e. administration results in delivery to a desired location in the subject where at least a portion of the composition delivered, are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous. For the delivery of cells, administration by injection or infusion is generally preferred.

The efficacy of a treatment comprising a composition described herein comprising the two guide ribonucleic acid (gRNAs) and the one or more DNA endonucleases (or a vector comprising a polynucleotide that encodes the gRNAs and the one or more DNA endonucleases) for the treatment of ALS can be determined by the skilled clinician. However, a treatment is considered “effective treatment,” if any one or all of the signs or symptoms of, as but one example, levels of hexanucleotide repeat-containing transcripts are altered in a beneficial manner (e.g., decreased by at least 10%), or other clinically accepted symptoms or markers of disease are improved or ameliorated. Efficacy can also be measured by failure of an individual to worsen as assessed by hospitalization or need for medical interventions (e.g., chronic obstructive pulmonary disease, or progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

It is contemplated that administration of a composition described herein ameliorates one or more symptoms associated with ALS by reducing the amount of hexanucleotide repeat in the individual. Early signs typically associated with ALS include for example, dementia, difficulty walking, weakness in the legs, hand weakness, clumsiness, slurring of speech, trouble swallowing, muscle cramps, twitching in the arms or shoulders or tongue, difficulty holding the head up or keeping good posture.

Kits

The present disclosure provides kits for carrying out the methods of the invention. A kit can include one or more of a guide RNA, and DNA endonuclease necessary to carry out the embodiments of the methods of the invention, or any combination thereof.

In some embodiments, a kit comprises: (1) a vector comprising a nucleotide sequence encoding a genome-targeting nucleic acid, and (2) a vector comprising a nucleotide sequence encoding the site-directed polypeptide or the site-directed polypeptide and (3) a reagent for reconstitution and/or dilution of the vector(s) and or polypeptide.

In some embodiments, a kit comprises: (1) a vector comprising (i) a nucleotide sequence encoding a genome-targeting nucleic acid, and (ii) a nucleotide sequence encoding the site-directed polypeptide and (2) a reagent for reconstitution and/or dilution of the vector.

In some embodiments of any of the above kits, the kit comprises a single-molecule guide genome-targeting nucleic acid. In some embodiments of any of the above kits, the kit comprises a double-molecule genome-targeting nucleic acid. In some embodiments of any of the above kits, the kit comprises two or more double-molecule guides or single-molecule guides. In some embodiments, the kits comprise a vector that encodes the nucleic acid targeting nucleic acid.

In some embodiments of any of the above kits, the kit can further comprise a polynucleotide to be inserted to effect the desired genetic modification.

Components of a kit may be in separate containers, or combined in a single container.

In some embodiments, a kit described above further comprises one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a kit can also include one or more components that may be used to facilitate or enhance the on-target binding or the cleavage of DNA by the endonuclease, or improve the specificity of targeting.

In addition to the above-mentioned components, a kit can further include instructions for using the components of the kit to practice the methods. The instructions for practicing the methods are generally recorded on a suitable recording medium. For example, the instructions may be printed on a substrate, such as paper or plastic, etc. The instructions may be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this embodiment is a kit that includes a web address where the instructions can be viewed and/or from which the instructions can be downloaded. As with the instructions, this means for obtaining the instructions can be recorded on a suitable substrate.

### Definitions

The term “comprising” or “comprises” is used in reference to compositions, methods, and respective component(s) thereof, that are essential to the invention, yet open to the inclusion of unspecified elements, whether essential or not.

The term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The singular forms “a,” “an,” and “the” include plural references, unless the context clearly dictates otherwise.

Certain numerical values presented herein are preceded by the term “about.” The term “about” is used to provide literal support for the numerical value the term “about” precedes, as well as a numerical value that is approximately the numerical value, that is the approximating unrecited numerical value may be a number which, in the context it is presented, is the substantial equivalent of the specifically recited numerical value. The term “about” means numerical values within +10% of the recited numerical value.

When a range of numerical values is presented herein, it is contemplated that each intervening value between the lower and upper limit of the range, the values that are the upper and lower limits of the range, and all stated values with the range are encompassed within the disclosure. All the possible sub-ranges within the lower and upper limits of the range are also contemplated by the disclosure.

### EXAMPLES

The invention will be more fully understood by reference to the following examples, which provide illustrative non-limiting embodiments of the disclosure.

### Example 1—SpCas9 Guide RNA Screening

To identify cell lines suitable for phenotype-based screening, two patient iPSC lines (ND50037 and CS52) and used for the experiments described herein. ND50037 has approximately 200-250 repeats as estimated by Southern Blotting. CS52 has an expanded allele with approximately 800 GGGGCC repeats.

Select SpCas9 gRNAs set forth in SEQ ID NOs: 1-14 were tested in a patient-iPSC cell line in a phenotype-based screen that used C9ORF72-derived transcripts measured by NanoString assay as a read-out. The goal of the phenotype-based screen was to identify guide pairs that reduce the levels of repeat-containing transcripts while preserving the expression of Exon1b containing transcripts as far as possible.

For the phenotype-based screen, three broad regions of the C9ORF72 locus (C9) were deleted: 1) 5′ flank of G4C2 repeats including Exon1a and upstream promoter region, 2) G4C2 repeats, and 3) CpG island to the 3′ flank of G4C2 repeats. G4C2 expanded repeats form secondary structures (G quadruplexes) and are difficult for enzymes to transcribe and amplify. Therefore, to avoid bias against repeat-containing transcripts, a NanoString assay (NanoString Technologies) was utilized to measure C9ORF72 transcripts since it does not rely on reverse transcription and amplification.

Briefly, the NanoString assay depends on capturing fragmented RNA molecules using a biotinylated capture probe and subsequently detecting this fragment using a reporter probe that binds immediately adjacent to the capture probe on the RNA fragment. A signal is detected only when both capture and reporter probes are bound to the same RNA fragment. The probes were ordered from Integrated DNA Technologies, Inc. and other assay reagents were purchased from NanoString technologies, Inc. The assay was performed as per manufacturer's (NanoString Tech.) instructions.

A NanoString assay was established to detect various transcripts generated from C9ORF72 locus (Donnelly et al., 2013, Haeusler et al., 2014; van Blitterswijk et al., 2015, Gendron et al., 2015). In the NanoString assay, levels of spliced transcripts with Exon1a, repeat-containing transcripts, and spliced transcripts with Exon1b were assessed. Exon1b containing transcripts are the predominant transcripts in cell types in both control and C9/ALS patient-derived IPSCs. The goal of the phenotype-based screen was to identify guide pairs that reduce the levels of repeat-containing transcripts while preserving the expression of Exon1b containing transcripts, as far as possible.

Sp Cas9 gRNAs (SEQ ID NOs: 1-9) were synthesized by in vitro transcription. Double stranded DNA ‘gene blocks’ were ordered from Integrated DNA Technologies, Inc. These gene blocks consist of sequence corresponding to T7 RNA polymerase promoter and the gRNA spacer sequence followed by the gRNA backbone sequence. The gene block was amplified by PCR and gRNA synthesis was performed by in vitro transcription using GeneArt Precision gRNA Synthesis Kit (Thermo Fisher Scientific) by following manufacturer's instructions. Alternatively, chemically modified gRNAs were purchased for studies with CS52 cell line.

The gRNAs were incubated with SpCas9 protein at room temperature for 15-20 minutes in the Lonza nucleofection buffer P3 to form a ribonucleoprotein complex (RNP) and this RNP was delivered to C9/ALS iPSCs by nucleofection (Lonza nucleofector device). 200 k cells were nucleofected with SpCas9/gRNA RNP at 1:3 ratio. Post nucleofection, cells were grown for 6 days before harvesting them for RNA isolation and NanoString assay. NanoString assays were performed as per instructions of the manufacturer.

Results of the NanoString assay indicated that (1) Exon1b containing transcripts are the predominant form in iPSCs; (2) Exon1b containing transcripts were downregulated in the C9ORF72 ALS patient-derived iPSC line tested (ND50037); and (3) repeat containing transcripts were upregulated in the tested C9ORF72 ALS patient-derived iPSC line compared to a control wildtype iPSC line (data not shown).

Multiple gRNA pairs were tested for each strategy and experiments were repeated three times. Unedited C9ORF72 ALS patient-derived iPSCs were included as controls in each experiment and the average counts for each transcript from these samples were used as 100%. Transcript counts from edited samples were normalized to respective counts seen in unedited control samples and averaged across three separate experiments. FXN, HPRT, TBP, TUBB, and CNOT10 transcripts were used to normalize RNA input across different samples.

Screening Results:

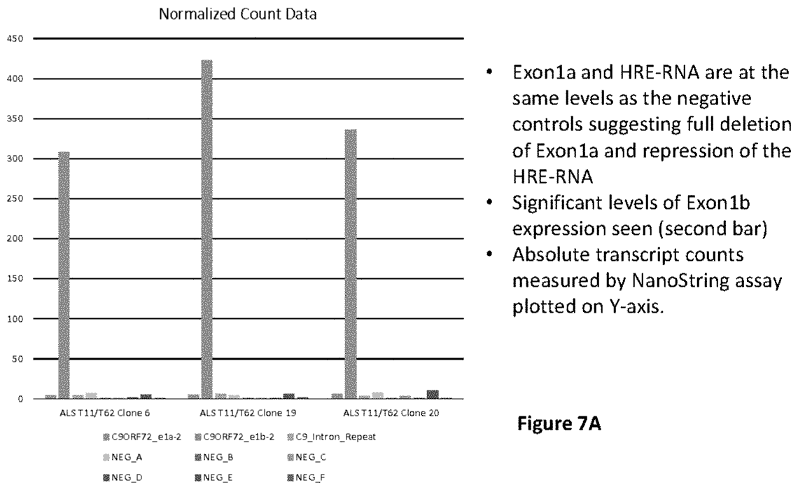

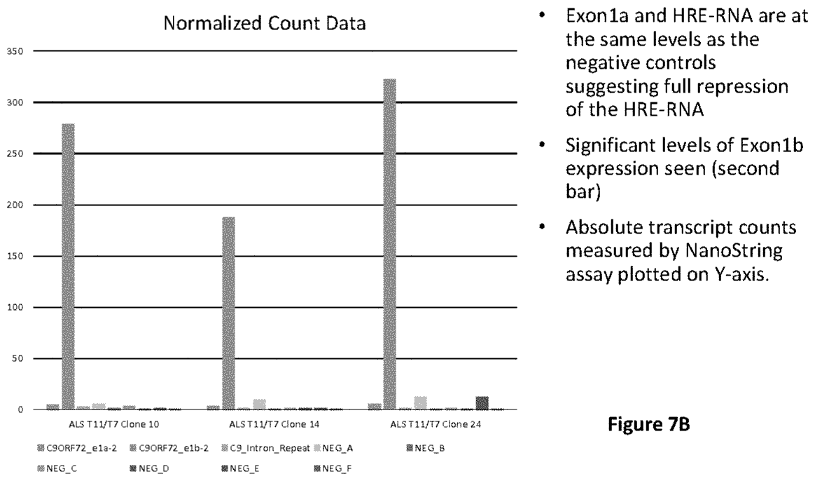

A total 27 gRNA pairs were tested, 14 pairs of which showed 40% reduction in repeat containing transcript levels (FIG. 7 and Table 2). Results are shown as expression of the gene as a percentage of the control. The experiments were repeated 3-4 times, where the standard deviations were not outside the normal range of such studies.

TABLE 2
ND50037 cell line study

| Sp | | | | C9ORF72_exon1a-2 | C9ORF72_Intron_Repeat |
|---|---|---|---|---|---|
| Pair# | Name | 20mer Spacer Sequence | PAM | (%) | (%) |
| 1 | T11 | TGTGCGAACCTTAATAGGGG, (SEQ ID NO: 1) | AGG | 26 | 49 |
| | T7 | CCAAGCGTCATCTTTTACGT, (SEQ ID NO: 2) | GGG | | |
| 2 | T11 | TGTGCGAACCTTAATAGGGG, (SEQ ID NO: 1) | AGG | 22 | 34 |
| | T118 | TGCGGTGCCTGCGCCCGCGG, (SEQ ID NO: 7) | CGG | | |
| 3 | T11 | TGTGCGAACCTTAATAGGGG, (SEQ ID NO: 1) | AGG | 39 | 90 |
| | T128 | GTACTGTGAGAGCAAGTAGT, (SEQ ID NO: 9) | GGG | | |
| 4 | T11 | TGTGCGAACCTTAATAGGGG, (SEQ ID NO: 1) | AGG | 50 | 55 |
| | T69 | GGTTGCGGTGCCTGCGCCCG, (SEQ ID NO: 6) | CGG | | |
| 5 | T17 | GACCCGCTCTGGAGGAGCGT, (SEQ ID NO: 8) | TGG | 47 | 55 |
| | T118 | TGCGGTGCCTGCGCCCGCGG, (SEQ ID NO: 7) | CGG | | |
| 6 | T11 | TGTGCGAACCTTAATAGGGG, (SEQ ID NO: 1) | AGG | 39 | 39 |
| | T5 | GAACTCAGGAGTCGCGCGCT, (SEQ ID NO: 15) | AGG | | |
| 7 | T11 | TGTGCGAACCTTAATAGGGG, (SEQ ID NO: 1) | AGG | 43 | 64 |
| | T62 | TGCTCTCACAGTACTCGCTG, (SEQ ID NO: 4) | AGG | | |
| 8 | T17 | GACCCGCTCTGGAGGAGCGT, (SEQ ID NO: 8) | TGG | 59 | 74 |
| | T7 | CCAAGCGTCATCTTTTACGT, (SEQ ID NO: 2) | GGG | | |
| 9 | T17 | GACCCGCTCTGGAGGAGCGT, (SEQ ID NO: 8) | TGG | 35 | 75 |
| | T128 | GTACTGTGAGAGCAAGTAGT, (SEQ ID NO: 9) | GGG | | |
| 10 | T17 | GACCCGCTCTGGAGGAGCGT, (SEQ ID NO: 8) | TGG | 57 | 67 |
| | T62 | TGCTCTCACAGTACTCGCTG, (SEQ ID NO: 4) | AGG | | |
| 11 | T17 | GACCCGCTCTGGAGGAGCGT, (SEQ ID NO: 8) | TGG | 48 | 125 |
| | T69 | GGTTGCGGTGCCTGCGCCCG, (SEQ ID NO: 6) | CGG | | |
| 12 | T3 | GCGTGTGCGAACCTTAATAG, (SEQ ID NO: 3) | GGG | 19 | 29 |
| | T118 | TGCGGTGCCTGCGCCCGCGG, (SEQ ID NO: 7) | CGG | | |
| 13 | T3 | GCGTGTGCGAACCTTAATAG, (SEQ ID NO: 3) | GGG | 29 | 99 |
| | T128 | GTACTGTGAGAGCAAGTAGT, (SEQ ID NO: 9) | GGG | | |
| 14 | T3 | GCGTGTGCGAACCTTAATAG, (SEQ ID NO:3) | GGG | 30 | 26 |
| | T5 | GAACTCAGGAGTCGCGCGCT, (SEQ ID NO: 5) | AGG | | |
| 15 | T3 | GCGTGTGCGAACCTTAATAG, (SEQ ID NO: 3) | GGG | 28 | 39 |
| | T69 | GGTTGCGGTGCCTGCGCCCG, (SEQ ID NO: 6) | CGG | | |
| 16 | T30 | CGCCAACGCTCCTCCAGAGC, (SEQ ID NO: 5) | GGG | 42 | 57 |
| | T7 | CCAAGCGTCATCTTTTACGT, (SEQ ID NO: 2) | GGG | | |
| 17 | T30 | CGCCAACGCTCCTCCAGAGC, (SEQ ID NO: 5) | GGG | 21 | 38 |
| | T118 | TGCGGTGCCTGCGCCCGCGG, (SEQ ID NO: 7) | CGG | | |
| 18 | T30 | CGCCAACGCTCCTCCAGAGC, (SEQ ID NO: 5) | GGG | 26 | 27 |
| | T5 | GAACTCAGGAGTCGCGCGCT, (SEQ ID NO: 15) | AGG | | |
| 19 | T30 | CGCCAACGCTCCTCCAGAGC, (SEQ ID NO: 5) | GGG | 33 | 36 |
| | T69 | GGTTGCGGTGCCTGCGCCCG, (SEQ ID NO: 6) | CGG | | |
| 20 | T7 | CCAAGCGTCATCTTTTACGT, (SEQ ID NO: 2) | GGG | 65 | 110 |
| | T128 | GTACTGTGAGAGCAAGTAGT, (SEQ ID NO: 9) | GGG | | |
| 21 | T128 | GTACTGTGAGAGCAAGTAGT, (SEQ ID NO: 9) | GGG | 60 | 53 |
| | T69 | GGTTGCGGTGCCTGCGCCCG, (SEQ ID NO: 6) | CGG | | |
| 22 | T132 | ATCCTGGCGGGTGGCTGTTT, (SEQ ID NO: 132) | GGG | 100 | 100 |
| | T44 | CTTTCGCCTCTAGCGACTGG, (SEQ ID NO: 13) | TGG | | |
| 23 | T132 | ATCCTGGCGGGTGGCTGTTT, (SEQ ID NO: 12) | GGG | 86 | 100 |
| | T51 | GCGAGGCCTCTCAGTACCCG, (SEQ ID NO: 16) | AGG | | |
| 24 | T132 | ATCCTGGCGGGTGGCTGTTT, (SEQ ID NO: 12) | GGG | 69 | 85 |
| | T9 | GGCTTCTGCGGACCAAGTCG, (SEQ ID NO: 14) | GGG | | |
| 25 | T5 | GAACTCAGGAGTCGCGCGCT, (SEQ ID NO: 15) | AGG | 48 | 236 |
| | T69 | GGTTGCGGTGCCTGCGCCCG, (SEQ ID NO: 6) | CGG | | |
| 26 | T3 | GCGTGTGCGAACCTTAATAG, (SEQ ID NO: 3) | GGG | 50 | 54 |
| | T62 | TGCTCTCACAGTACTCGCTG, (SEQ ID NO: 4) | AGG | | |
| 27 | T30 | CGCCAACGCTCCTCCAGAGC, (SEQ ID NO: 5) | GGG | 39 | 54 |
| | T62 | TGCTCTCACAGTACTCGCTG, (SEQ ID NO: 4) | AGG | | |

TABLE 3
CS52 cell line study.

| Sp | | | | C9ORF72_exon1a-2 | C9ORF72_Intron_Repeat |
|---|---|---|---|---|---|
| Pair# | Name | 20mer Spacer Sequence | PAM | (%) | (%) |
| 1 | T11 | TGTGCGAACCTTAATAGGGG, (SEQ ID NO: 1) | AGG | 23 | 48 |
| | T7 | CCAAGCGTCATCTTTTACGT, (SEQ ID NO: 2) | GGG | | |
| 2 | T11 | TGTGCGAACCTTAATAGGGG, (SEQ ID NO: 1) | AGG | 23 | 144 |
| | T62 | TGCGGTGCCTGCGCCCGCGG, (SEQ ID NO: 4) | CGG | | |

Deletion of regions upstream of G4C2 repeats (that included Exon1a) resulted in a reduction in repeat-containing transcripts. As presented in the Tables 1 and 2 above, the use of gRNA pairs T11 and T7 (SEQ ID NOs: 1 and 2, respectively), T11 and T118 (SEQ ID NOs: 1 and 7, respectively), T11 and T69 (SEQ ID NOs: 1 and 6, respectively), T17 and T118 (SEQ ID NOs: 8 and 7, respectively), T11 and T5 (SEQ ID NOs: 1 and 15, respectively), T3 and T118 (SEQ ID Nos: 3 and 7, respectively), T3 and T5 (SEQ ID NOs: 3 and 15, respectively), T3 and T69 (SEQ ID NOs: 3 and 6, respectively), T30 and T7 (SEQ ID NOs: 5 and 2, respectively), T30 and T118 (SEQ ID NOs: 5 and 7, respectively), T30 and T5 (SEQ ID NOs: 5 and 15, respectively), T30 and T69 (SEQ ID NOs: 5 and 6, respectively), T128 and T69 (SEQ ID NOs: 9 and 6, respectively), and T30 and T62 (SEQ ID NOs: 5 and 4, respectively) resulted in a reduction of at least 40% in repeat-containing transcripts, as measured by C9ORF72 intron repeat transcript expression.

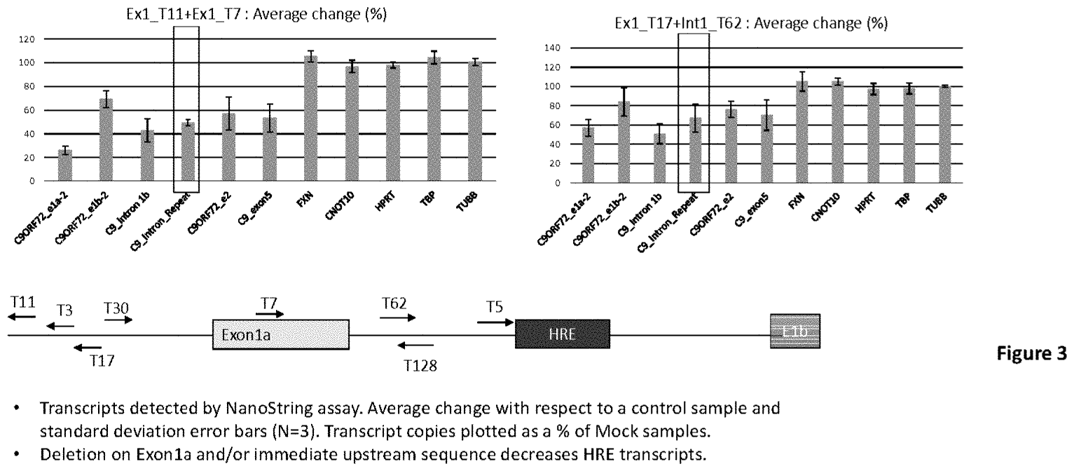

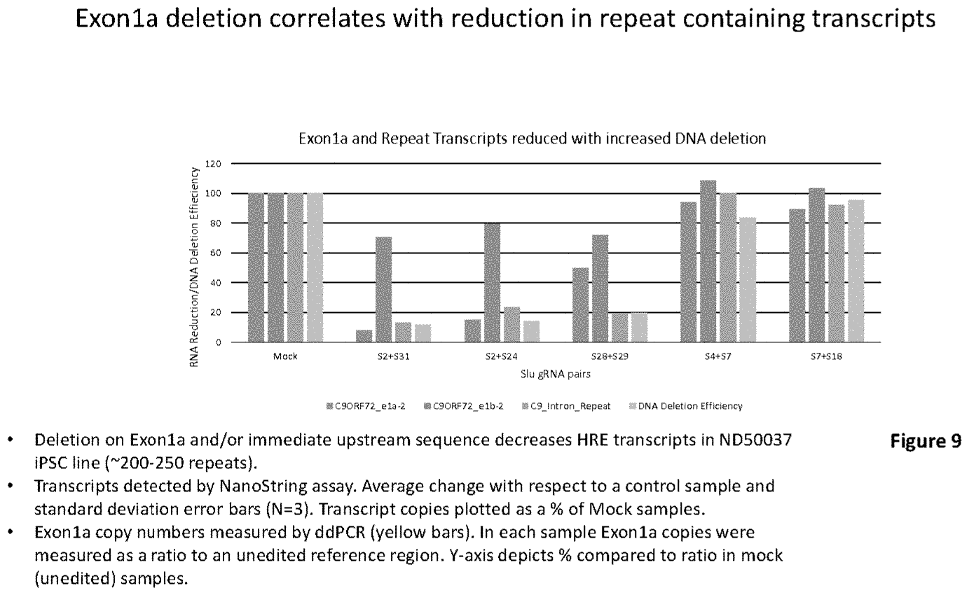

As seen from FIG. 9, the results further exemplify that reduction of at least 40% of repeat-containing transcripts is achieved using a CRISPR/Cas9 system wherein a first DSB is within nucleotides 1801-1970 of SEQ ID NO: 42 (Target region 1 of FIG. 9) and a second DSB is within nucleotides 2189-2326 of SEQ ID NO: 42 (Target region 3 of FIG. 9). Reduction of at least 40% of repeat-containing transcripts is also achieved using a CRISPR/Cas9 system wherein a first DSB is within nucleotides 1801-1970 of SEQ ID NO: 42 (Target region 1 of FIG. 9) and a second DSB is within nucleotides 2384-2900 of SEQ ID NO: 42 (Target region 4 of FIG. 9). Reduction of at least 40% of repeat-containing transcripts is also achieved using a CRISPR/Cas9 system wherein a first DSB is within nucleotides 1801-1970 of SEQ ID NO: 42 (Target region 1 of FIG. 9) and a second DSB is within nucleotides 2051-2156 of SEQ ID NO: 42 (Target region 2 of FIG. 9). Data from the gRNA pairs—T11/T7 (SEQ ID NOs: 1 and 2, respectively) and T17/T62 (SEQ ID NOs: 8 and 4, respectively), are shown in FIG. 3. These two gRNA pairs caused ˜40%-50% reduction in repeat-containing transcripts (fourth bar from the left on both graphs).

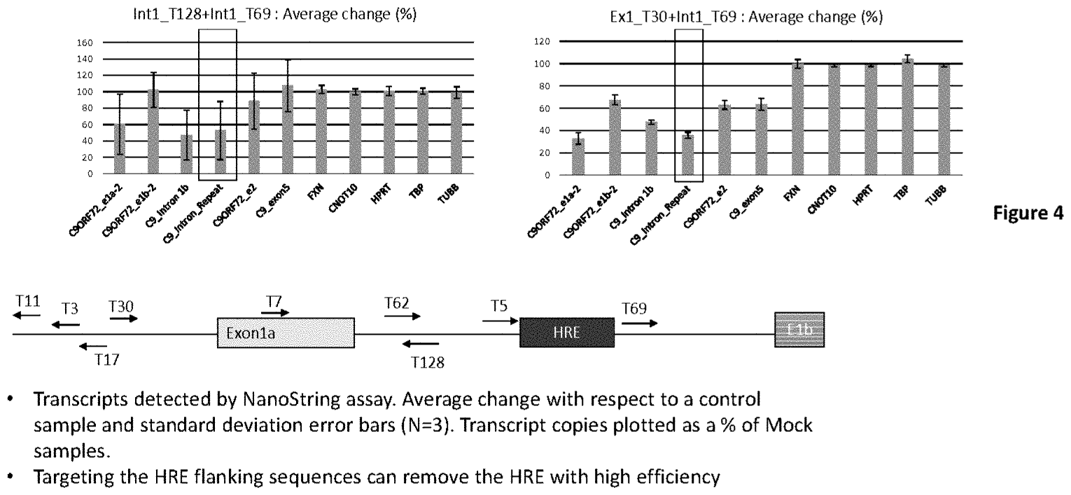

Data from two gRNA pairs T128/T69 (SEQ ID NOs: 9 and 6, respectively) and T30/T69 (SEQ ID NOs: 5 and 6, respectively) that delete the repeats are shown in FIG. 4. T30/T69 also deletes Exon1a, in addition to the G4C2 repeats. Both of these guide pairs appear to reduce the levels of repeat RNA significantly.

Guide pairs T132/T44 (SEQ ID NOs: 12 and 13) and T132/T9 (SEQ ID NOs: 12 and 14) delete a potential regulatory region on the 3′ flank of the G4C2 repeats. This region appears to not regulate the expression of repeat-containing transcripts (FIG. 5).

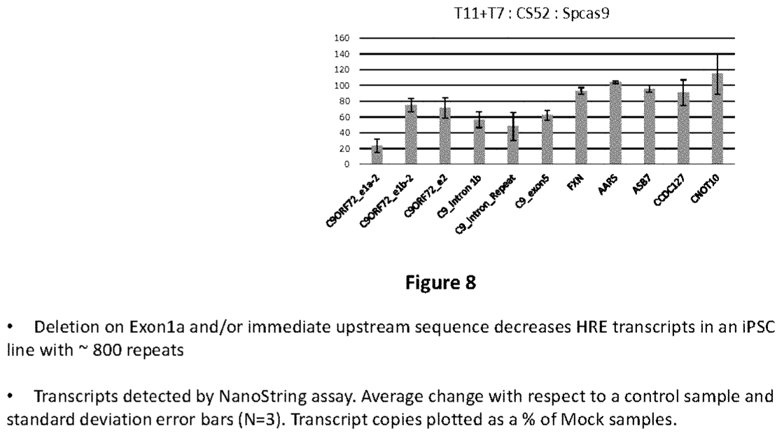

The nucleofection and screening assay described in this Example was repeated in a CS52 iPSC cell line with guide pair T11/T7 (SEQ ID NOs: 1 and 2, respectively). Data shows that this guide pair caused ˜40%-50% reduction in repeat-containing transcripts (FIG. 8, fifth bar from the left and Table 3 shown above).

### Example 2—Derivation of Edited Isogenic iPSC Lines

Isogenic edited patient-iPSC lines are valuable to understand the effects of specific gene edits and can be differentiated into relevant cell types (e.g. spinal motor neurons) for in vitro proof-of-concept experiments. In this Example, the effect of removing Exon1a and flanking sequences on the expression of repeat-containing transcripts was investigated at the level of clonal cell populations.

Isogenic clonal lines were generated from an ALS patient-derived-iPSC line (ND50037) after editing with gRNA pairs that delete Exon1a either partially or fully (T11/T62 and T11/T7) as described in Example 1. Briefly, 1 million cells were nucleofected using the same experimental conditions as the bulk nucleofection described above in Example 1. After several days single cells were sorted into individual wells using the Hana single cell sorter from Namocell following the manufacturers instructions. Clones were grown and passaged until NanoString analysis could be performed as described above.

The generated lines were tested for C9ORF72 transcript expression by the NanoString assay as described above in Example 1. As shown in FIGS. 8A and 8B, the level of C9ORF72 repeat containing transcripts (third bar from the left in each clone tested) in the tested clones was close to signal seen with NanoString negative controls. The negative controls are probes designed against sequences not seen in human transcriptomes and indicate baseline non-specific signal. This data suggests that deleting Exon1a/part of Exon1a and upstream sequence from a C9ORF72 allele caused a complete loss of repeat expression from that allele and that these clones are homozygous for Exon1a sequence deletion. Significant levels of Exon1b expression was also observed (second bar form the left in each clone tested).

### Example 3—SluCas9 Guide RNA Screening

Select gRNAs set forth in SEQ ID NOs: 17-41 were tested in a patient-iPSC cell line (ND50037) and a CS52 iPSC cell line (CY52CPYiALS) in a phenotype-based screen that used C9ORF72-derived transcripts measured by Nanostring assay as a read-out. SluCas9 gRNA pairs that delete the 5′ flank of G4C2 repeats including exon 1a and the upstream promoter region were tested.

NanoString Assays were conducted with cell lysates or purified gRNAs. gRNAs were extracted using the RNEasy RNA extraction kit (QIAGEN) according to the manufacturer's instructions, were assessed. Chemically modified gRNAs were purchased from Synthego. Most samples were lysed using Cells-Ct lysis buffer from Thermofisher, according to the manufacturer's instructions. The gRNAs were incubated with SluCas9 protein at room temperature for 15-20 minutes in the Lonza nucleofection buffer P3 to form a ribonucleoprotein complex (RNP) and this RNP was delivered to C9/ALS iPSCs by nucleofection (Lonza nucleofector device). 200 k cells were nucleofected with SluCas9/gRNA RNP at 1:3 ratio. Post nucleofection, cells were grown for 6 days before harvesting them for RNA isolation and NanoString assay. Nanostring assays were performed as per the manufacturer's instructions.

Unedited C9ORF72 ALS patient-derived iPSCs were included as controls in each experiment and the average counts for each transcript from these samples were used as 100%. Transcript counts from edited samples were normalized to respective counts seen in unedited control samples and averaged across three separate experiments. FXN, HPRT, TBP, TUBB, and CNOT10 transcripts were used to normalize RNA input across different samples.

Screening Results:

A total 21 gRNA pairs were tested and results are shown below in Table 4. Results are shown as expression of the gene as a percentage of the control. The experiments were repeated 1-3 times, where the standard deviations were not outside the normal range of such studies.

TABLE 4
ND50037 cell line study

| Slu | | | | C9ORF72_exon1a-2 | C9ORF72_Intron_Repeat |
|---|---|---|---|---|---|
| Pair# | Name | 22mer Spacer Sequence | PAM | (%) | (%) |
| 1 | S3 | CGAACCTTAATAGGGGAGGCTG, (SEQ ID NO: 17) | CTGG | 53 | 140 |
| | S26 | CTTGCTCTCACAGTACTCGCTG, SEQ ID NO: 18) | AGGG | | |
| 2 | S3 | CGAACCTTAATAGGGGAGGCTG, (SEQ ID NO: 17) | CTGG | 59 | 64 |
| | S20 | CTGCCCGGTTGCTTCTCTTTTG, (SEQ ID NO: 20) | GGGG | | |
| 3 | S2 | TTCTTTTATCTTAAGACCCGCT, (SEQ ID NO: 20) | CTGG | 11 | 26 |
| | S24 | ACTTGCTCTCACAGTACTCGCT, (SEQ ID NO: 21) | GAGG | | |
| 4 | S2 | TTCTTTTATCTTAAGACCCGCT, (SEQ ID NO: 20) | CTGG | 10 | 24 |
| | S31 | CTAGCAAGAGCAGGTGTGGGTT, (SEQ ID NO: 22) | TAGG | | |
| 5 | S15 | ATTGCGCCAACGCTCCTCCAGA, (SEQ ID NO: 23) | GCGG | 16 | 66 |
| | S22 | GAGTACTGTGAGAGCAAGTAGT, SEQ ID NO: 24) | GGGG | | |
| 6 | S14 | GAAGACGATTTCGTGGTTTTGA, (SEQ ID NO: 25) | ATGG | 23 | 99 |
| | S22 | GAGTACTGTGAGAGCAAGTAGT, (SEQ ID NO: 24) | GGGG | | |
| 7 | S17 | TTTTATCTTAAGACCCGCTCTG, (SEQ ID NO: 26) | GAGG | 44 | 48 |
| | S26 | CTTGCTCTCACAGTACTCGCTG, (SEQ ID NO: 18) | AGGG | | |
| 8 | S17 | TTTTATCTTAAGACCCGCTCTG, (SEQ ID NO: 26) | GAGG | 68 | 74 |
| | S20 | CTGCCCGGTTGCTTCTCTTTTG, (SEQ ID NO: 19) | GGGG | | |
| 9 | S16 | TAAGACCCGCTCTGGAGGAGCG, (SEQ ID NO: 27) | TTGG | 46 | 74 |
| | S30 | CGGGGTCTAGCAAGAGCAGGTG, (SEQ ID NO: 28) | TGGG | | |
| 10 | S32 | TTGCGCCAACGCTCCTCCAGAG, (SEQ ID NO: 29) | CGGG | 46 | 69 |
| | S31 | CTAGCAAGAGCAGGTGTGGGTT, (SEQ ID NO: 22) | TAGG | | |
| 11 | S28 | TTAATAGGGGAGGCTGCTGGAT, (SEQ ID NO: 31) | CTGG | 47 | 26 |
| | S29 | GCGGGGTCTAGCAAGAGCAGGT, (SEQ ID NO: 40) | GTGG | | |
| 12 | S1 | GCGTGTGCGAACCTTAATAGGG, (SEQ ID NO: 41 | GAGG | 42 | 57 |
| | S22 | GAGTACTGTGAGAGCAAGTAGT, (SEQ ID NO: 24) | GGGG | | |
| 13 | S2 | TTCTTTTATCTTAAGACCCGCT, (SEQ ID NO: 20) | CTGG | 39 | 50 |
| | S9 | GCGAGTACTGTGAGAGCAAGTA, (SEQ ID NO: 34) | GTGG | | |
| 14 | S3 | CGAACCTTAATAGGGGAGGCTG, (SEQ ID NO: 17) | CTGG | 48 | 87 |
| | S5 | ACACCAAGCGTCATCTTTTACG, (SEQ ID NO: 32) | TGGG | | |
| 15 | S3 | CGAACCTTAATAGGGGAGGCTG, (SEQ ID NO: 17) | CTGG | 31 | 54 |
| | S6 | CCGCCCACGTAAAAGATGACGC, (SEQ ID NO: 33) | TTGG | | |
| 16 | S3 | CGAACCTTAATAGGGGAGGCTG, (SEQ ID NO: 17) | CTGG | 58 | 94 |
| | S9 | GCGAGTACTGTGAGAGCAAGTA, (SEQ ID NO: 34) | GTGG | | |
| 17 | S7 | CCAAGCGTCATCTTTTACGTGG, (SEQ ID NO: 37) | GCGG | 81 | 106 |

The nucleofection and screening assay described in) this Example was repeated twice in a CS52 iPSC cell line with 3 guide pairs (S2/S24, S2/S31; S2/S5, S2/S6, S2/S9, S28/S29). The results are provided below in Table 5.

TABLE 5

| Slu | | | | C9ORF72_exon1a-2 | C9ORF72_Intron_Repeat |
|---|---|---|---|---|---|
| Pair# | Name | 22mer Spacer Sequence | PAM | (%) | (%) |
| 1 | S2 | TTCTTTTATCTTAAGACCCGCT, SEQ ID NO: 20 | CTGG | 12 | 17 |
| | S24 | ACTTGCTCTCACAGTACTCGCT, SEQ ID NO: 21 | GAGG | | |
| 2 | S2 | TTCTTTTATCTTAAGACCCGCT, SEQ ID NO: 20 | CTGG | 7 | 21 |
| | S31 | CTAGCAAGAGCAGGTGTGGGTT, SEQ ID NO: 22 | TAGG | | |
| 3 | S2 | TTCTTTTATCTTAAGACCCGCT, SEQ ID NO: 20 | CTGG | 16 | 33 |
| | S6 | CCGCCCACGTAAAAGATGACGC, SEQ ID NO: 33 | TTGG | | |

As presented in the Tables 4 and 5 above, the use of gRNA pairs S2 and S24 (SEQ ID NOs: 20 and 21, respectively), S2 and S31 (SEQ ID NOs: 20 and 22, respectively), S17 and S26 (SEQ ID NOs: X and X, respectively), S28 and S29 (SEQ ID NOs: 26 and 40, respectively), 51 and S22 (SEQ ID Nos: 41 and 24, respectively), S2 and S9 (SEQ ID Nos: 20 and 34, respectively), S3 and S6 (SEQ ID NOs: 17 and 33, respectively), and S2 and S6 (SEQ ID NOs: 20 and 33, respectively) resulted in a reduction of at least 40% in repeat-containing transcripts, as measured by C9ORF72 intron repeat transcript expression.

As seen from FIG. 10, the results further exemplify that reduction of at least 40% of repeat-containing transcripts is achieved using a CRISPR/Cas9 system wherein a first DSB is within nucleotides 1801-1970 of SEQ ID NO: 42 (Target region 1 of FIG. 10) and a second DSB is within nucleotides 2189-2326 of SEQ ID NO: 42 (Target region 3 of FIG. 10). Reduction of at least 40% of repeat-containing transcripts is also achieved using a CRISPR/Cas9 system wherein a first DSB is within nucleotides 1801-1970 of SEQ ID NO: 42 (Target region 1 of FIG. 10) and a second DSB is within nucleotides 2051-2156 of SEQ ID NO: 42 (Target region 2 of FIG. 10).

The location of the cut site for SpCas9 gRNA T5 overlaps with the NanoString probe used to detect repeat-containing transcripts. Similarly, the location of the cut site for SluCas9 gRNAs S20, S29, S30, and S31 overlaps with the NanoString probe used to detect Exon 1a transcripts. In the experiments described above that use one or more of these gRNAs as part of a gRNA pair, it is theoretically possible that some of the reduction in probe counts observed after gene editing are caused due to overlapping indels and are not due to true deletions.

In order to further confirm that reduction in repeat-containing transcripts with these gRNA pairs, a Droplet Digital PCR (ddPCR) assay was developed to directly measure deletions in Exon 1a. DNA was extracted and purified from cell pellets using the QIAGEN DNEasy Blood and Tissue Kit according to the manufacturer's instructions. Quality and concentration were assessed on the NanoDrop 2000 spectrophotometer. Prior to ddPCR assay, up to 1 ug of DNA was digested for at least 3 hours with CviQI restriction enzyme from New England BioLabs. After digestion, ddPCR assay was performed on the DNA following the instructions from Bio-Rad. Droplet generation was done on the Bio-Rad Automated droplet generator. PCR reaction was performed on the Bio Rad thermocycler and finally read on the Bio-Rad QX200 droplet reader. Analysis of results was performed on QuantaSoft Analysis Pro software from Bio-Rad. To determine deletion efficiency, a ratio between the target amplicon and the reference amplicon was calculated. This is a loss of signal assay. A reduction in target amplification indicates successful gene editing. The primers and probes used are presented in Table 6.

TABLE 6
Target (C9ORF72 Exon1a) Primers and Probes

| | | SEQ ID NO:. |
|---|---|---|
| Forward Primer | GCTAGCCTCGTGAGAAAACG | 43 |
| Reverse Primer | CTCTTTCCTAGCGGGACACC | 44 |
| Probe* (FAM | CATCGCA+CATA+GAA+AA+ | 45 |
| Fluorophore) | CA+GACA+GAC | |

*The C9ORF72 target probe contains locked nucleic acids (LNA) to increase the meltingtemperature of the probe. The nucleic acid preceding the “+” is the LNA.

The assay was used to test samples from the gene editing experiments performed in the ND50037 patient iPSC line. As shown in FIG. 9, it was observed that the vast majority of C9ORF72 alleles had deletions in Exon 1a when cells were transfected with either guide pairs S2 and S31 or guide pairs S2 and S24 with 92% and 85% reduction respectively. This correlates well with the significant reduction in repeat-containing transcripts observed in these samples using the NanoString assay.

While the present disclosure provides descriptions of various specific aspects for the purpose of illustrating various aspects of the present invention and/or its potential applications, it is understood that variations and modifications will occur to those skilled in the art. Accordingly, the invention or inventions described herein should be understood to be at least as broad as they are claimed, and not as more narrowly defined by particular illustrative aspects provided herein.

Any patent, publication, or other disclosure material identified herein is incorporated by reference into this specification in its entirety unless otherwise indicated, but only to the extent that the incorporated material does not conflict with existing descriptions, definitions, statements, or other disclosure material expressly set forth in this specification. As such, and to the extent necessary, the express disclosure as set forth in this specification supersedes any conflicting material incorporated by reference. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

1-37. (canceled)

38. A method for editing a C9ORF72 gene in a human cell by gene editing comprising delivering to the cell one or more CRISPR systems comprising one or more guide ribonucleic acids (gRNAs) and one or more site-directed deoxyribonucleic acid (DNA) endonucleases, and wherein the one or more site-directed DNA enconucleases are Cas9 endonucleases that effect double-stranded breaks (DSBs) within a region of the C9ORF72 gene comprising nucleotides 1801-2900 of SEQ ID NO: 42 that causes a permanent deletion of the hexanucleotide repeat of the C9ORF72 gene.

39. (canceled)

40. The method of claim 38, wherein the region of the C9ORF72 gene comprises nucleotides 1801-1970 of SEQ ID NO: 42, or nucleotides 2051-2156 of SEQ ID NO: 42, or nucleotides 2189-2326 of SEQ ID NO: 42, or nucleotides 2384-2900 of SEQ ID NO: 42.

41-43. (canceled)

44. The method of claim 38, wherein
(a) a first DSB is within nucleotides 1801-1970 of SEQ ID NO: 42 and a second DSB is within nucleotides 2051-2156 of SEQ ID NO: 42
(b) a first DSB is within nucleotides 1801-1970 of SEQ ID NO: 42 and a second DSB is within nucleotides 2189-2326 of SEQ ID NO: 42; or
(c) a first DSB is within nucleotides 1801-1970 of SEQ ID NO: 42 and a second DSB is within nucleotides 2384-2900 of SEQ ID NO: 42.

45-46. (canceled)

47. The method of claim 38, wherein the one or more gRNAs are:
(a) SEQ ID NO: 1 and SEQ ID NO: 2 (T1 and T7);
(b) SEQ ID NO: 1 and SEQ ID NO: 7 (T1 and T118);
(c) SEQ ID NO: 1 and SEQ ID NO: 6 (T1 and T69);
(d) SEQ ID NO: 8 and SEQ ID NO: 7 (T17 and T118);
(e) SEQ ID NO: 1 and SEQ ID NO: 15 (T1 and T5);
(f) SEQ ID NO: 3 and SEQ ID NO: 7 (T3 and T118);
(g) SEQ ID NO: 3 and SEQ ID NO: 15 (T3 and T5);
(h) SEQ ID NO: 3 and SEQ ID NO: 6 (T3 and T69);
(i) SEQ ID NO: 5 and SEQ ID NO: 2 (T30 and T7);
(j) SEQ ID NO: 5 and SEQ ID NO: 7 (T30 and T118);
(k) SEQ ID NO: 5 and SEQ ID NO: 15 (T30 and T5);
(l) SEQ ID NO: 5 and SEQ ID NO: 6 (T30 and T69);
(m) SEQ ID NO: 9 and SEQ ID NO: 6 (T128 and T69); or
(n) SEQ ID NO: 5 and SEQ ID NO: 4 (T30 and T62).

48. The method of claim 38, wherein the one or more gRNAs are:
(a) SEQ ID NO: 20 and SEQ ID NO: 21 (S2 and S24);
(b) SEQ ID NO: 20 and SEQ ID NO: 22 (S2 and S31);
(c) SEQ ID NO: 26 and SEQ ID NO: 18 (S17 and S26);
(d) SEQ ID NO: 26 and SEQ ID NO: 29 (S28 and S29);
(e) SEQ ID NO: 41 and SEQ ID NO: 24 (51 and S22);
(f) SEQ ID NO: 20 and SEQ ID NO: 34 (S2 and S9);
(g) SEQ ID NO: 17 and SEQ ID NO: 33 (S3 and S6); or
(h) SEQ ID NO: 20 and SEQ ID NO: 33 (S2 and S6).

49. The method of claim 38, wherein the one or more gRNAs are:
(a) SEQ ID NO: 20 and SEQ ID NO: 21 (S2 and S24),
(b) SEQ ID NO: 20 and SEQ ID NO: 22 (S2 and S31),
(c) SEQ ID NO: 20 and SEQ ID NO: 33 (S2 and S6),
(d) SEQ ID NO: 20 and SEQ ID NO: 34 (S2 and S9),
(e) SEQ ID NO: 17 and SEQ ID NO: 33 (S3 and S6),
(f) SEQ ID NO: 26 and SEQ ID NO: 18 (S17 and S26), or
(g) SEQ ID NO: 31 and SEQ ID NO: 40 (S28 and S29).

50. A composition comprising one or more guide ribonucleic acids (gRNAs) comprising
(a) a spacer sequence selected from the nucleotide sequence set forth in SEQ ID NOs.: 1-41;
(b) a spacer sequence set forth in one or more of SEQ ID NOs: 1, 2, 3, 4, 5, 6, 7, 8, 9, and 15; or
(c) a spacer sequence set forth in one or more of SEQ ID NOs: 1, 2, 3, 4, 5, 6, 7, 8, 9, 15, 17, 18, 20, 21, 26, 31, 33, 34, and 40.

51-54. (canceled)

55. A recombinant expression vector comprising a nucleotide sequence that encodes the one or more gRNAs of claim 50.

56-83. (canceled)

84. The composition of claim 50, wherein the one or more gRNAs are:
(a) SEQ ID NO: 1 and SEQ ID NO: 2 (T1 and T7);
(b) SEQ ID NO: 1 and SEQ ID NO: 7 (T1 and T118);
(c) SEQ ID NO: 1 and SEQ ID NO: 6 (T1 and T69);
(d) SEQ ID NO: 8 and SEQ ID NO: 7 (T17 and T118);
(e) SEQ ID NO: 1 and SEQ ID NO: 15 (T1 and T5);
(f) SEQ ID NO: 3 and SEQ ID NO: 7 (T3 and T118);
(g) SEQ ID NO: 3 and SEQ ID NO: 15 (T3 and T5);
(h) SEQ ID NO: 3 and SEQ ID NO: 6 (T3 and T69);
(i) SEQ ID NO: 5 and SEQ ID NO: 2 (T30 and T7);
(j) SEQ ID NO: 5 and SEQ ID NO: 7 (T30 and T118);
(k) SEQ ID NO: 5 and SEQ ID NO: 15 (T30 and T5);
(l) SEQ ID NO: 5 and SEQ ID NO: 6 (T30 and T69);
(m) SEQ ID NO: 9 and SEQ ID NO: 6 (T128 and T69); or
(n) SEQ ID NO: 5 and SEQ ID NO: 4 (T30 and T62).

85. The composition of claim 50, wherein the one or more gRNAs are:
(a) SEQ ID NO: 20 and SEQ ID NO: 21 (S2 and S24);
(b) SEQ ID NO: 20 and SEQ ID NO: 22 (S2 and S31);
(c) SEQ ID NO: 26 and SEQ ID NO: 18 (S17 and S26);
(d) SEQ ID NO: 26 and SEQ ID NO: 29 (S28 and S29);
(e) SEQ ID NO: 41 and SEQ ID NO: 24 (51 and S22);
(f) SEQ ID NO: 20 and SEQ ID NO: 34 (S2 and S9);
(g) SEQ ID NO: 17 and SEQ ID NO: 33 (S3 and S6); or
(h) SEQ ID NO: 20 and SEQ ID NO: 33 (S2 and S6).

86. The composition of claim 50, wherein the one or more gRNAs are:
(a) SEQ ID NO: 20 and SEQ ID NO: 21 (S2 and S24),
(b) SEQ ID NO: 20 and SEQ ID NO: 22 (S2 and S31),
(c) SEQ ID NO: 20 and SEQ ID NO: 33 (S2 and S6),
(d) SEQ ID NO: 20 and SEQ ID NO: 34 (S2 and S9),
(e) SEQ ID NO: 17 and SEQ ID NO: 33 (S3 and S6),
(f) SEQ ID NO: 26 and SEQ ID NO: 18 (S17 and S26), or
(g) SEQ ID NO: 31 and SEQ ID NO: 40 (S28 and S29).


SEQUENCE LISTING (45 sequences)

SEQ ID NO: 1 - DNA, 20 bp, artificial sequence
  Synthetic
tgtgcgaaccttaatagggg

SEQ ID NO: 2 - DNA, 20 bp, artificial sequence
  Synthetic
ccaagcgtcatcttttacgt

SEQ ID NO: 3 - DNA, 20 bp, artificial sequence
  Synthetic
gcgtgtgcgaaccttaatag

SEQ ID NO: 4 - DNA, 20 bp, artificial sequence
  Synthetic
tgctctcacagtactcgctg

SEQ ID NO: 5 - DNA, 20 bp, artificial sequence
  Synthetic
cgccaacgctcctccagagc

SEQ ID NO: 6 - DNA, 20 bp, artificial sequence
  Synthetic
ggttgcggtgcctgcgcccg

SEQ ID NO: 7 - DNA, 20 bp, artificial sequence
  Synthetic
tgcggtgcctgcgcccgcgg

SEQ ID NO: 8 - DNA, 20 bp, artificial sequence
  Synthetic
gacccgctctggaggagcgt

SEQ ID NO: 9 - DNA, 20 bp, Artificial sequence
  Synthetic
gtactgtgagagcaagtagt

SEQ ID NO: 10 - DNA, 20 bp, artificial sequence
  Synthetic
acagagtagacccttggttg

SEQ ID NO: 11 - DNA, 20 bp, artificial sequence
  Synthetic
ggttttgtacagtcccctct

SEQ ID NO: 12 - DNA, 20 bp, artificial sequence
  Synthetic
atcctggcgggtggctgttt

SEQ ID NO: 13 - DNA, 20 bp, artificial sequence
  Synthetic
ctttcgcctctagcgactgg

SEQ ID NO: 14 - DNA, 20 bp, artificial sequence
  Synthetic
ggcttctgcggaccaagtcg

SEQ ID NO: 15 - DNA, 20 bp, artificial sequencre
  Synthetic
gaactcaggagtcgcgcgct

SEQ ID NO: 16 - DNA, 20 bp, artificial sequence
  Synthetic
gcgaggcctctcagtacccg

SEQ ID NO: 17 - DNA, 22 bp, artificial sequence
  Synthetic
cgaaccttaataggggaggctg

SEQ ID NO: 18 - DNA, 22 bp, artificial sequence
  Synthetic
cttgctctcacagtactcgctg

SEQ ID NO: 19 - DNA, 22 bp, artificial sequence
  Synthetic
ctgcccggttgcttctcttttg

SEQ ID NO: 20 - DNA, 22 bp, artificial sequence
  Synthetic
ttcttttatcttaagacccgct

SEQ ID NO: 21 - DNA, 22 bp, artificial sequence
  Synthetic
acttgctctcacagtactcgct

SEQ ID NO: 22 - DNA, 22 bp, artificial sequence
  Synthetic
ctagcaagagcaggtgtgggtt

SEQ ID NO: 23 - DNA, 22 bp, artificial sequence
  Synthetic
attgcgccaacgctcctccaga

SEQ ID NO: 24 - DNA, 22 bp, artificial sequence
  Synthetic
gagtactgtgagagcaagtagt

SEQ ID NO: 25 - DNA, 22 bp, artificial sequence
  Synthetic
gaagacgatttcgtggttttga

SEQ ID NO: 26 - DNA, 22 bp, artificial sequence
  Synthetic
ttttatcttaagacccgctctg

SEQ ID NO: 27 - DNA, 22 bp, artificial sequence
  Synthetic
taagacccgctctggaggagcg

SEQ ID NO: 28 - DNA, 22 bp, artificial sequence
  Synthetic
cggggtctagcaagagcaggtg

SEQ ID NO: 29 - DNA, 22 bp, artificial sequence
  Synthetic
ttgcgccaacgctcctccagag

SEQ ID NO: 30 - DNA, 22 bp, artificial sequence
  Synthetic
gcggggtctagcaagagcaggt

SEQ ID NO: 31 - DNA, 22 bp, artificial sequence
  Synthetic
ttaataggggaggctgctggat

SEQ ID NO: 32 - DNA, 22 bp, artificial sequence
  Synthetic
acaccaagcgtcatcttttacg

SEQ ID NO: 33 - DNA, 22 bp, artificial sequence
  Synthetic
ccgcccacgtaaaagatgacgc

SEQ ID NO: 34 - DNA, 22 bp, artificial sequence
  Synthetic
gcgagtactgtgagagcaagta

SEQ ID NO: 35 - DNA, 22 bp, artificial sequence
  Synthetic
acttgctctcacagtactcgct

SEQ ID NO: 36 - DNA, 22 bp, artificial sequence
  Synthetic
agcgggtcttaagataaaagaa

SEQ ID NO: 37 - DNA, 22 bp, artificial sequence
  Sythetic
ccaagcgtcatcttttacgtgg

SEQ ID NO: 38 - DNA, 22 bp, artificial sequence
  Synthetic
cacaccaagcgtcatcttttac

SEQ ID NO: 39 - DNA, 22 bp, artificial sequence
  Synthetic
cccggttgcttctcttttgggg

SEQ ID NO: 40 - DNA, 22 bp, artificial sequence
  Synthetic
gcggggtctagcaagagcaggt

SEQ ID NO: 41 - DNA, 22 bp, artificial sequence
  Synthetic
gcgtgtgcgaaccttaataggg

SEQ ID NO: 42 - DNA, 2900 bp, Homo sapiens
tcaggtgagacttgggactttggacttttgaatgaatgctggatcgagttaagactttggggaactgttggtaaggcacgacagtattttgcaatatgagaaggacattagatttgggaggggccagagttggaataacatggtttggatctctgtccccacccaaatctcatgttcaactgtaatccccagtgttggaggttgggcctggtgggaggtgagtggattatggggtggcttctaatggttttgtacagtcccctcttggtactatatagtgagttctgacaagatctagttgtttaaacgtatgtagcacctcccatttctctcttcccccagttcctgccatgtgaagtctggggtctccctatgccttccatcatgattttaagttccctatggcctgcccagaagctgatccagccatgcttcttgtacagcctgcagaactgtgagccattaaacttttctttataaattacccagtttcagttatttctttatagcagtgtaagaatggactaacacaattattaacgctagtcctcatgttgtacattaaatctctagatgtattagacgtaactgcaactttgtaccctaccctacaattttctttccccccaagccccccaaccaagggtctactctgtttctataaattcagttgttttttaattccacgtataagtgaagtacaactcagtgtagaaacttggtaaatgctagctacttgttataagctgtcagtcaaaataaaaatacagagatgaatctctaaattaagtgatttatttgggaagaaagaattgcaattagggcatacatgtagatcagatggtcttcggtatatccacacaacaaagaaaagggggaggttttgttaaaaaagagaaatgttacatagtgctctttgagaaaattcattggcactattaaggatctgaggagctggtgagtttcaactggtgagtgatggtggtagataaaattagagctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcggg

SEQ ID NO: 43 - DNA, 20 bp, artificial sequence
  Synthetic
gctagcctcgtgagaaaacg

SEQ ID NO: 44 - DNA, 20 bp, artificial sequence
  Synthetic
ctctttcctagcgggacacc

SEQ ID NO: 45 - DNA, 25 bp, artificial sequence
  Synthetic
catcgcacatagaaaacagacagac